(19) United States
(12) Patent Application Publication
(10) Pub. No.: US 2004/0241153 A1
(43) Pub. Date: Dec. 2, 2004

(54) Methods of generating human cd4+ th1 cells

(76) Inventors: Daniel H. Fowler, Bethesda, MD (US); Jeanne Hou, Bethesda, MD (US); Unsu Jung, Ashburn, VA (US); Ronald E. Gress, Gaithersburg, MD (US); Bruce Levine, Cherry Hill, NJ (US); Carl June, Merion Station, PA (US)

Correspondence Address: KLARQUIST SPARKMAN, LLP, 121 SW SALMON STREET, SUITE 1600, PORTLAND, OR, 97204, US

(21) Appl. No.: 10/488,196

(22) PCT Filed: Aug. 29, 2002
(86) PCT No.: PCT/US02/27824

Related U.S. Application Data
(60) Provisional application No. 60/316,854, filed on Aug. 31, 2001.

(51) Int. Cl. A61K 45/00; C12N 5/08

(52) U.S. Cl. 424/93.71; 435/372

(57) ABSTRACT

Methods are provided for producing a population of substantially purified CD4+ Th1 lymphocytes. The method includes stimulating a population of substantially purified CD4+ T cells isolated from a subject by contacting the population with an anti-CD3 monoclonal antibody and an antibody that specifically binds to a T cell costimulatory molecule in the presence of a Th1 supportive environment to form a stimulated population of T cells. The stimulated population of CD4+ T cells is allowed to proliferate in a Th1 supportive environment. In one example, the Th1 supportive environment includes at least 20 IU/ml of IL-2, for example about 1000 I.U./ml of IL-2, and a neutralizing amount of an IL-4, an IL-13, and/or an IL-4/IL-13 neutralizing agent. In other examples, the supportive environment further includes at least 1 ng/ml of IL-12, for example about 2.5 ng/ml of IL-12. Purified populations of Th1 cells are disclosed herein, as are methods for their use.

### CROSS REFERENCE TO RELATED APPLICATION

This application claims the benefit of priority to U.S. Provisional Application 60/316,854 filed Aug. 31, 2001, herein incorporated by reference in its entirety.

### FIELD

This application relates to the methods for purification of CD4⁺ Th1 cells, to substantially purified populations of CD4⁺ Th1 cells, and to therapeutic uses of purified CD4⁺ Th1 cells.

### BACKGROUND

The T lymphocyte (“T cell”) is a key cell type in the human cellular immune system, providing both function and biochemical control. T cells are classified based on which cell surface receptors and cytokines they express. The expression of cell surface receptors CD4 and/or CD8 are generally used to define two broad classes of T cells; these cell surface receptors are involved in recognizing antigens presented to the T cells by antigen presenting cells (APC). Certain mature T cells express only CD4 but not CD8 (termed CD4+ cells), while other mature T cells express CD8 but not CD4 (termed CD8⁺ cells).

CD8⁺ cells recognize peptide antigens that are presented on MHC class I molecules. Upon activation by an APC (which involves binding of both a stimulatory antigen and a costimulatory ligand), a CD8⁺ T cell matures into a cytotoxic T cell, which has defined functions and characteristics. CD4⁺ T cells recognize antigens that are presented on MHC class II molecules. When activated by an APC, CD4⁺ T cells can differentiate into T helper (Th) cells. Th cells have been divided into subclasses based on their cytokine secretion profiles. Th1 cells secrete a specific set of cytokines, including interferon-γ (IFN-γ), interleukin-12 (IL-12), interleukin-2 (IL-2), interferon-γ, and lymphotoxin, and activate the cellular immunity processes (such as macrophage activation and induction of IgG antibodies by B cells). Th2 cells secrete different cytokines (particularly IL-4, IL-5 and IL-10), and mediate humoral immunity and allergic reactions.

CD4⁺ Th1 and Th2 cells are differentially implicated in immune responses to different diseases and other immune conditions. Recently, techniques have been developed that enable the expansion of mixed populations of T cells in vitro, involving activation of lymphocytes using “artificial APCs” (see, for instance, Garlie et al., 1999; U.S. Pat. No. 5,858,358; and published PCT Application Nos. US94/06255 and US94/13782). However, obtaining purified populations of CD4⁺ Th1 and Th2 cells separately would be beneficial both for studying the role of these cells, and for treating various disorders.

A Th1 CD4 response is associated with a favorable immune response in the setting of serious infectious disease. The association of Th1 CD4 immunity with improvement in a subject's response to infection has been observed with bacterial infections, viral infections including HIV, and fungal infections, including tuberculosis and aspergillosis. Low levels of immune Th1 cells may reduce a subject's ability to fight cancer or serious infections. Therefore, identification of methods which allow for growing and administering large numbers of Th1 cells as an immune therapy for cancer and infectious diseases, is needed.

### SUMMARY

Disclosed herein are novel methods for generating CD4⁺ Th1 cells and the purification of these cells. Specifically, culture conditions are disclosed herein that allow Th1 cells to be selectively propagated in vitro. The ability to grow and administer substantially pure populations of Th1 cells also represents a new therapy to enhance the immune system of a subject. Thus, the ability to grow Th1 cells represents methods for treating infections and/or cancer, methods for enhancing a vaccine response (i.e. as an adjuvant for a vaccine) and for improving autologous stem cell transplantation.

In one embodiment, a method is provided for producing a population of substantially purified CD4⁺ Th1lymphocytes. The method includes stimulating a population of substantially purified CD4⁺ T cells isolated from a subject by contacting the population with an anti-CD3 monoclonal antibody and an antibody that specifically binds to a T cell costimulatory molecule in the presence of a Th1 supportive environment to form a stimulated population of T cells. In one embodiment, the stimulated population of CD4⁺ T cells is allowed to proliferate in a Th1 supportive environment.

Purified populations of Th1 cells are disclosed herein, as are methods for their use.

The foregoing and other objects, features, and advantages of the methods and cells described herein will become more apparent from the following detailed description of several embodiments, which proceeds with reference to the accompanying figures.

### BRIEF DESCRIPTION OF THE FIGURES

FIG. 1 is a graph of the T cell yield of human CD4⁺ cells cultured under conditions designed to induce Th1 or Th2 cell growth. Similar numbers of cells were obtained under the two sets of culture conditions.

FIG. 2 are bar graphs showing the cytokines produced when cells were cultured under conditions designed to generate either Th1 or Th2 cells. The “<” symbol denotes that the cytokine content was below the detection limit for the assay.

FIG. 3 is a bar graph showing the amount of IL-13 secretion by various purified CD4+ cells. The data shown are the mean and the standard error of the mean for four separate donors for each of the culture conditions.

FIG. 4 are bar graphs showing the cytokines produced when CD4+ cells which are further purified into a CD4⁺RA⁺ T cell subset (RA) or the CD4⁺RO⁺ T cell subset (RO) then cultured under conditions designed to generate either Th1 or Th2 cells. The “<” symbol denotes that the cytokine content was below the detection limit for the assay.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. As used herein and in the appended claims, the singular forms “a” or “an” or “the” include plural references unless the context clearly dictates otherwise. For example, reference to “a cytokine” includes a plurality of such cytokines and reference to “the antibody” includes reference to one or more antibodies and equivalents thereof known to those skilled in the art, and so forth.

Unless explained otherwise, all technical and scientific terms used herein have the same meaning as commonly understood to one of ordinary skill in the art to which this disclosure belongs.

Animal: Living multicellular vertebrate organisms, a category which includes, for example, mammals and birds.

Antibody: Immunoglobulin molecules and immunologically active portions of immunoglobulin molecules, i.e., molecules that contain an antigen binding site which specifically binds (immunoreacts with) an antigen. In one embodiment the antigen is CD3. In another embodiment, the antigen is a co-stimulatory molecule (e.g. CD28).

A naturally occurring antibody (e.g., IgG) includes four polypeptide chains, two heavy (H) chains and two light (L) chains inter-connected by disulfide bonds. However, it has been shown that the antigen-binding function of an antibody can be performed by fragments of a naturally occurring antibody. Thus, these antigen-binding fragments are also intended to be designated by the term “antibody”. Examples of binding fragments encompassed within the term antibody include (i) an Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-6, 1989) which consists of a VH domain; (v) an isolated complimentarity determining region (CDR); and (vi) an F(ab′)₂ fragment, a bivalent fragment comprising two Fab fragments linked by a disulfide bridge at the hinge region. Furthermore, although the two domains of the Fv fragment are coded for by separate genes, a synthetic linker can be made that enables them to be made as a single protein chain (known as single chain Fv (scFv); Bird et al., Science 242:423-6, 1988; and Huston et al., Proc. Natl. Acad Sci. 85:5879-83, 1988) by recombinant methods. Such single chain antibodies are also included.

In one embodiment, antibody fragments for use in T cell expansion are those which are capable of crosslinking their target antigen, e.g., bivalent fragments such as F(ab′)₂ fragments. Alternatively, an antibody fragment which does not itself crosslink its target antigen (e.g., a Fab fragment) can be used in conjunction with a secondary antibody which serves to crosslink the antibody fragment, thereby crosslinking the target antigen. Antibodies can be fragmented using conventional techniques and the fragments screened for utility in the same manner as described for whole antibodies. An antibody is further intended to include bispecific and chimeric molecules that specifically bind the target antigen.

“Specifically binds” refers to the ability of individual antibodies to specifically immunoreact with an antigen, such as a T cell surface molecule. The binding is a non-random binding reaction between an antibody molecule and an antigenic determinant of the T cell surface molecule. The desired binding specificity is typically determined from the reference point of the ability of the antibody to differentially bind the T cell surface molecule and an unrelated antigen, and therefore distinguish between two different antigens, particularly where the two antigens have unique epitopes. An antibody that specifically binds to a particular epitope is referred to as a “specific antibody”.

Antigen: A substance capable of being the target of inducing a specific immune response.

Anti-microbial agent: A compound (or combination of compounds) that destroys an infectious agent, or prevents the infectious agent from multiplying. Examples include, but are not limited to antibiotics (such as penicillin and ampicillin), anti-viral compounds (such as AZT and protease inhibitors), anti-fungal compounds (such as amphotericin B), and anti-parasitic compounds (such as pentamidine).

B Cell: A lymphocyte, a type of white blood cell (leukocyte), that develops into a plasma cell, which produces antibodies.

Bone marrow transplant (BMT): The intravenous infusion of bone marrow. The marrow may be from a previously harvested and stored self-donation (autologous transplant), from a living donor other than the recipient (allogeneic transplant), or from an identical twin donor (syngeneic transplant). Used to treat malignancies such as leukemia, lymphoma, myeloma, and selected solid tumors, as well as nonmalignant conditions such as aplastic anemia, immunologic deficiencies, and inborn errors of metabolism.

Cancer: Malignant neoplasm that has undergone characteristic anaplasia with loss of differentiation, increase rate of growth, invasion of surrounding tissue, and is capable of metastasis.

Chemotherapy: In cancer treatment, chemotherapy refers to the administration of one or a combination of compounds to kill or slow the reproduction of rapidly multiplying cells. In rheumatology, chemotherapy is often designed to decrease the abnormal behavior of cells, rather than kill cells. The amount of chemotherapeutic agent used for rheumatic or autoimmune conditions are usually lower than the doses used for cancer treatment. Chemotherapuetic agents include those known by those skilled in the art, including, but not limited to: 5-fluorouracil (5-FU), azathioprine, cyclophosphamide, antimetabolites (such as Fludarabine), antineoplastics (such as Etoposide, Doxorubicin, methotrexate, and Vincristine), carboplatin, cis-platinum and the taxanes, such as taxol.

Chemotherapy-resistant disease: A disorder that is not responsive to administration of a chemotherapeutic agent.

Comprises: A term that means “including.” For example, “comprising A or B” means including A or B, or both A and B, unless clearly indicated otherwise.

Costimulator of a T cell: Although stimulation of the TCR/CD3 complex (or CD2 molecule) is required for delivery of a primary activation signal in a T cell, a number of molecules on the surface of T cells, termed accessory or costimulatory molecules, have been implicated in regulating the transition of a resting T cell to blast transformation, and subsequent proliferation and differentiation (T cell stimulation). Thus, in addition to the primary activation signal provided through the TCR/CD3 complex, induction of T cell responses requires a second, costimulatory signal. A costimulator of a T cell includes, but is not limited to CD28, inducible costimulatory molecule (ICOS), 4-1BB receptor (CDw137), lymphocyte function-associated antigen-1 (LFA-1), CD30, or CD154.

One such costimulatory or accessory molecule, CD28, is understood to initiate or regulate a signal transduction pathway that is distinct from those stimulated by the TCR complex. Other specific, non-limiting examples of co-stimulatory molecules are inducible costimulatory molecule (ICOS), 4-1BB receptor (CDw137), lymphocyte function-associated antigen-1 (LFA-1), CD30, or CD154 (see Salomon and Bluestone, Ann. Rev. Immunol. 19:225-52, 2001).

Thus, to induce an activated population of T cells to proliferate (i.e., a population of T cells that has received a primary activation signal) an accessory molecule on the surface of the T cell (e.g. CD28), is stimulated with a ligand which binds the accessory molecule. In one embodiment, stimulation of the accessory molecule is acheived by contacting an activated population of T cells with a ligand that binds to the accessory molecule, or with an antibody that specifically binds the accessory molecule.

In one embodiment, activation of CD4⁺T cells with an anti-CD3 antibody and an anti-CD28 antibody results in selective proliferation of CD4⁺ T cells. An anti-CD28 monoclonal antibody or fragment thereof capable of cross-linking the CD28 molecule, or a natural ligand for CD28 (e.g., a member of the B7 family of proteins, such as B7-1(CD80) and B7-2 (CD86) (Freedman et al. 1987. J. Immunol. 137:3260-7; Freeman et al. 1989. J. Immunol. 143:2714-22; Freeman et al. 1991. J. Exp. Med. 174:625-31; Freeman et al. 1993. Science 262:909-11; Azuma et al. 1993. Nature 366:76-9; Freeman et al. 1993. J. Exp. Med. 178:2185-92) can be used to induce stimulation of the CD28 molecule. In addition, binding homologues of a natural ligand, whether native or synthesized by chemical or recombinant technique, can also be used. Ligands useful for stimulating an accessory molecule can be used in soluble form or immobilized on a solid phase surface as described herein. Anti-CD28 antibodies of fragments thereof useful in stimulating proliferation of CD4⁺ T cells include monoclonal antibody 9.3, an IgG2a antibody (Jeffery Ledbetter, Bristol Myers Squibb Corporation, Seattle, Wash.), monoclonal antibody KOLT-2, an IgG1 antibody, 15E8, an IgG1 antibody, 248.23.2, an IgM antibody and EX5.3D10, an IgG2a antibody (see U.S. Pat. No. 5,858,358).

Cytokine/Interleukin (IL): A generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Many growth factors and cytokines act as cellular survival factors by preventing programmed cell death. Cytokines and interleukins include both naturally occurring peptides and variants that retain full or partial biological activity. Although specific cytokines/interleukins are described in the specification, they are not limited to the specifically disclosed peptides.

Enhance: To improve the quality, amount, or strength of something. In one embodiment, a therapy enhances the immune system if the immune system is more effective at fighting infection or tumors. In addition, or alternatively, a therapy enhances the immune system if the number of lymphocytes increases subsequent to the therapy. In a particular embodiment, a therapy enhances the immune system if the number of Th1 cells in the subject increases subsequent to the therapy, thereby enhancing a type I cytokine profile. Such enhancement can be measured using the methods disclosed herein, for example determining the level of type I cytokines produced using an ELISA, or determining the increase in lymphocytes using flow cytometry.

In another embodiment, a therapy enhances a vaccine response. In one embodiment, a therapy enhances a vaccine response if the number of antibodies produced increases, and/or if the antibodies produces are more effective at fighting infection or tumors. Such enhancement can be measured using any bioassay known in the art, for example, an ELISA assay.

Immobilized: Bound to a surface, such as a solid surface. A solid surface can be polymeric, such as polystyrene or polypropylene. In one embodiment, the solid surface is the bottom surface of a flask or a tissue culture plate. In another embodiment, the solid surface is in the form of a bead. A specific, non-limiting example of a bead is Tosylated magnetic beads (Dynal). Methods of immobilizing antibodies and peptides on a solid surface can be found in WO 94/29436, and U.S. Pat. No. 5,858,358.

Immuno-deplete: To decrease the number of lymphocytes, such as CD4⁺ and/or CD8⁺ cells, in a subject.

Immuno-depleting agent: One or more compounds, when administered to a subject, result in a decrease in the number of cells of the immune system (such as lymphocytes) in the subject. Examples include, but are not limited to, chemotherapeutic agents, monoclonal antibodies, and other therapies disclosed in EXAMPLE 7.

Infection: Invasion and multiplication of microorganisms in a subject, which may cause local cellular injury due to competitive metabolism, toxins, intracellular replication, and/or antigen-antibody response.

Infectious disease. Any disease caused by an infectious agent. Examples of infectious agents include, but are not limited to: bacteria, viruses, fungi and parasites. In a particular embodiment, it is a disease caused by at least one type of infectious agent. In another embodiment, it is a disease caused by at least two different types of infectious agents. Infectious diseases can affect any body system, be acute (short-acting) or chronic (long-acting), occur with or without fever, strike any age group, and overlap each other.

Examples of diseases caused by bacterial infections include, but are not limited to: gastroenteritis (caused by salmonella, shigella, campylobacter, E. coli, and/or yersinia); gonorrhea; Legionnaires' disease (caused by Legionella pneumophila); lyme disease (caused by Borrelia burgdorferi); Pertussis (whooping cough; caused by Bordetella pertussis); pharyngitis (caused by group A streptococcus and Corynebacterium diphtheriae); bacterial pneumonia (caused by Streptococcus pneumoniae, Mycoplasma pneumoniae, Chlamydia pneumoniae, Klebsiella pseudomonas, and Staphylococcus aureus); sinusitis (caused by Staphylococcus aureus); Streptococcal (strep) infection (caused by Streptococcus); syphilis (caused by Treponema pallidum); and tuberculosis.

Examples of diseases resulting from viral infections include, but are not limited to: AIDS (caused by HIV); chicken pox/shingles (caused by Varicella zoster virus, VZV); encephalitis; influenza; hepatitis A, B or C; herpes (caused by HSV-1 or HSV-2); infectious mononucleosis (caused by Epstein-Barr virus); measles; rabies; rubella; and viral meningitis.

Examples of fungal infections include but are not limited to: aspergillosis; thrush (caused by Candida albicans); cryptococcosis (caused by Cryptococcus); and histoplasmosis.

Examples of diseases caused by parasitic infections include, but are not limited to: amebiasis; ascariasis; giardiasis malaria; pinworms; tapeworms; and toxoplasmosis.

Interferon-gamma (IFN-γ): Includes both naturally occurring peptides, as well as IFN-γ fragments and variants that retain full or partial IFN-γ biological activity. IFN-γ is a dimeric protein glycosylated at two sites with subunits of 146 amino acids. Murine and human IFN-γ have approximately 40% sequence homology at the protein level. The human IFN-γ gene is approximately 6 kb, contains four exons and maps to chromosome 12q24.1. At least six variants of naturally occurring IFN-γ have been described, and differ from each other by variable lengths of the C-terminal ends.

IFN-γ is produced mainly by T-cells and natural killer cells activated by antigens, mitogens, or alloantigens. It is produced by lymphocytes expressing the surface antigens CD4 and CD8. The synthesis of IFN-γ is induced, among other things, by IL2, βFGF, and EGF. The synthesis of IFN-γ is inhibited by 1-alpha, 25-Dihydroxy vitamin D3, dexamethasone and CsA (Cyclosporin A).

In Th cells, IL2 induces the synthesis of IFN-γ and other cytokines. IFN-γ also stimulates the expression of Ia antigens on the cell surface, the expression of CD4 in T helper cells, and the expression of high-affinity receptors for IgG in myeloid cell lines, neutrophils, and monocytes.

IFN-γ can be detected by immunoassay. A specific ELISA test allows detection of individual cells producing IFN-γ. Minute amounts of IFN-γ can be detected indirectly by measuring IFN-induced proteins such as Mx protein. The induction of the synthesis of IP-10 has been used also to measure IFN-γ concentrations. One bioassay employs induction of indoleamine 2,3-dioxygenase activity in 2D9 cells. A sensitive radioreceptor assay is also available.

Interleukin (IL)-2: Includes both naturally occurring IL-2 peptides, as well as IL-2 fragments and variants that retain full or partial IL-2 biological activity. A protein of 133 amino acids (15.4 kDa) with a slightly basic pI. IL-2 does not display sequence homology to any other factors. Murine and human IL-2 display a homology of approximately 65%. IL2 is synthesized as a precursor protein of 153 amino acids with the first 20 amino terminal amino acids functioning as a hydrophobic secretory signal sequence. The protein contains a single disulfide bond (positions Cys58/105) essential for biological activity. Naturally occurring IL-2 is O-glycosylated at threonine at position 3. However, variants exist with different molecular masses and charges are due to variable glycosylation. Non-glycosylated IL-2 is also biologically active.

The human IL-2 gene contains four exons. The IL-2 gene maps to human chromosome 4q26-28, while the mouse gene maps to murine chromosome 3. The homology of murine and human IL-2 is 72% at the nucleotide level in the coding region.

Mouse and human IL-2 both cause proliferation of T-cells of the homologous species at high efficiency. Human IL-2 also stimulates proliferation of mouse T-cells at similar concentrations, whereas mouse IL-2 stimulates human T-cells at a lower (sixfold to 170-fold) efficiency. IL-2 is a growth factor for all subpopulations of T-lymphocytes. It is an antigen-unspecific proliferation factor for T-cells that induces cell cycle progression in resting cells, and allows clonal expansion of activated T-lymphocytes. Due to its effects on T-cells and B-cells, IL-2 is considered to be a central regulator of immune responses (Waguespack et al., Brain. Res. Bull. 34: 103-9, 1994)

IL-2 can be assayed in bioassays employing cell lines that respond to the factor (e.g., ATH8, CT6, CTLL-2, FDCPmix, HT-2, NKC-3, TALL-103). Specific ELISA assays for IL-2 and enzyme immunoassays for the soluble receptor are also available. An alternative detection method is reverse transcriptase polymerase chain reaction (RT-PCR) (Brandt et al. 1986. Lymphokine Research 5: S35-S42; Lindqvist et al. 1988. J Immunol. Meth. 113: 231-5).

IL-4: Includes both naturally occurring IL-4 peptides, as well as IL-4 fragments and variants that retain full or partial IL-4 biological activity. IL-4 is a protein produced mainly by a subpopulation of activated T-cells (CD4⁺Th2 cells). IL-4 is a 129 amino acid protein (20 kDa) synthesized as a precursor containing a hydrophobic secretory signal sequence of 24 amino acids. IL-4 is glycosylated at two arginine residues (positions 38 and 105) and contains six cysteine residues involved in disulfide bond formation. Some glycosylation variants of IL-4 have been described that differ in their biological activities. Murine and human IL-4 proteins only diverge at positions 91-128.

The human IL-4 gene contains four exons and has a length of approximately 10 kb. It maps to chromosome 5q23-31, while the murine gene maps to chromosome 11. At the nucleotide level the human and the murine IL-4 gene display approximately 70 percent homology.

The biological activities of IL-4 are species-specific; mouse IL-4 is inactive on human cells and human IL-4 is inactive on murine cells. IL-4 promotes the proliferation and differentiation of activated B-cells, the expression of class II MHC antigens, and of low affinity IgE receptors in resting B-cells. In addition, IL-4 is known to enhance expression of class II MHC antigens on B-cells. This cytokine also can promote the B-cells' capacity to respond to other B-cell stimuli and to present antigens for T-cells.

The classical detection method for IL-4 is a B-cell costimulation assay measuring the enhanced proliferation of stimulated purified B-cells. IL-4 can be detected also in bioassays, employing IL4-responsive cells (e.g. BALM-4, BCL1, CCL-185, CT.4S, amongst others). A specific detection method for human IL-4 is the induction of CD3 in a number of B-cell lines with CD23 detected either by flow-through cytometry or by a fluorescence immunoassay.

An alternative detection method is RT-PCR (for review see: Boulay and Paul. 1992. Cur. Opin. Immunol. 4: 294-8; Paul and Ohara. 1987. Ann. Rev. Immunol. 5: 429-59). Total RNA is isolated with Trizol-LS (Life Technologies) according to manufacturer's instructions. Using the cDNA Cycle kit for RT-PCR (Invitrogen), three micrograms of each RNA are reverse transcribed into cDNA. The cDNA is quantified utilizing the CytoXpress Quantitative PCR kit for human IL-4 (Biosource International, Camarillo, Calif.).

IL-4 neutralizing agent: An agent which decreases the biological activity of IL-4, for example to an IL-4 activity level below that which can be detected using a standard immunoassay. Such agents can thus can be used to inhibit IL-4 activity. Examples of such agents, include, but are not limited to anti-IL-4 antibodies and soluble IL-4 receptor (Immunex). Particular examples include monoclonal IL-4 antibodies. Anti-human IL-4 antibodies and methods of making are known (for example see U.S. Pat. Nos. 5,863,537; 5,705,154; and U.S. Pat. No. 5,597,710 all to Daile et al. and 5,041,38 to Abrams et al.). In a particular example, an IL-4 neutralizing agent is an IL4/IL-13 Trap (Regeneron, Tarrytown, N.Y.) that binds to and neutralizes both IL-4 and IL-13.

IL-5: Includes both naturally occurring IL-5 peptides, as well as IL-5 fragments and variants that retain full or partial IL-5 biological activity. Murine IL-5 cDNA encodes a protein of 113 amino acids, while the human protein is 115 amino acids. The biologically active form of IL-5 is an N-glycosylated antiparallel homodimer linked by disulfide bonds. Monomeric forms are biologically inactive. Variable molecular masses of the native protein are caused by heterogeneous glycosylation. Non-glycosylated IL-5 is also biologically active.

Murine and human IL-5 protein sequences are approximately 70% identical. While murine and human IL-5 have the same specific activity on human cells, murine IL-5 is about 50-100-fold more active on murine cells than human IL-5. The C-terminus of the protein is responsible for the species specificity.

IL-5 is a specific hematopoietic growth factor responsible for the growth and differentiation of eosinophils. IL-5 promotes the growth of immature hematopoietic progenitor cells and strongly stimulates the proliferation, activation, and differentiation of eosinophilic granulocytes. IL-5 also promotes the generation of cytotoxic T-cells from thymocytes. In thymocytes, IL-5 induces the expression of high affinity IL-2 receptors.

IL-10: Includes both naturally occurring IL-10 peptides, as well as IL-10 fragments and IL-10 variants that retain full or partial IL-10 biological activity. IL-10 is a homodimeric protein with subunits having 160 amino acids. Human IL-10 shows 73% amino acid homology with murine IL-10 at the protein level, and 81% homology at the nucleotide level. Human IL-10 contains four exons and maps to chromosome 1.

In humans IL-10 is produced, for example, by activated CD8 (+)peripheral blood T-cells and by Th2 cells. IL-10 is produced by murine T-cells (Th2 but not Th1 cells) following their stimulation by lectins.

IL-10 inhibits the synthesis of a number of cytokines such as IFNγ, IL2 and TNF-β in Th1 subpopulations of T-cells but not of Th2 cells. This activity is antagonized by IL-4. In humans, IL-10 is produced by, and down-regulates the function of Th1 and Th2 cells. In human monocytes, IFN-γ and IL-10 antagonize each other's production and function. IL-10 is a physiologic antagonist of IL-12.

IL-10 also inhibits mitogen- or anti-CD3-induced proliferation of T-cells in the presence of accessory cells and reduces the production of IFN-γ and IL-2. Exogenous IL-2 and IL-4 inhibit the proliferation-inhibitory effect but do not influence the production of IFN-γ. In LPS-stimulated macrophages, IFN-γ increases the synthesis of IL-6 by inhibiting the production of IL-10. IL-10 appears to be responsible for most or all of the ability of Th2 supernatants to inhibit cytokine synthesis by Th1 cells.

Several methods can be used to detect IL-10, including, but not limited to: ELISA; using the murine mast cell line D36 can be used to bioassay human IL-10; and flow cytometry.

IL-12: Includes both naturally occurring IL-12 peptides, as well as IL-12 fragments and variants that retain full or partial IL-12 biological activity. IL-12 is a heterodimeric 70 kDa glycoprotein consisting of a 40 kDa subunit (40 kDa subunit, 306 amino acids; 10 percent carbohydrate) and a 35 kDa subunit (p35, 197 amino acids; 20 percent carbohydrate) linked by disulfide bonds.

The gene encoding the p40 subunit of IL-12 (IL-12B) maps to human chromosome 5q31-q33 in the same region that also harbors other cytokine genes. The gene encoding the p35 subunit of IL-12 (IL-12A) maps to human chromosome 3p12-q13.2. The expression of the two genes is regulated independently of each other.

IL-12 is secreted by peripheral lymphocytes after induction. It is produced mainly by B-cells and to a lesser extent by T-cells. The most powerful inducers of IL-12 are bacteria, bacterial products, and parasites. IL-12 is produced after stimulation with phorbol esters or calcium ionophore by human B-lymphoblastoid cells. IL-12 activates NK-cells positive for CD56, and this activity is blocked by antibodies specific for TNF-alpha.

IL-12 can be detected by assaying its activity as a NKSF (natural killer cell stimulatory factor) or a CLMF (cytotoxic lymphocyte maturation factor).

IL-13: Includes both naturally occurring IL-13 peptides, as well as IL-13 fragments and variants that retain full or partial IL-13 biological activity. Human IL-13 is expressed in activated T-helper cells and T-cells expressing CD8. Human and the murine IL-13 proteins share 58% sequence identity, and the cDNAs are 66% identical. Several isoform variants of human IL13 exist, which contain Gly or Asp at position 61 and an insertion of Gln at position 98.

The receptors for IL-13 and IL-4 share a common component, which is the common gamma subunit found also in the IL-2 receptor. Antibodies against IL-4 receptor also block the activities of IL-13. IL-13 also uses the one of the signaling molecules that is used also by IL-4 (IL-4-STAT). IL-13 competes for IL-4 binding. An IL-4 variant, Y124D, in which Tyr124 is substituted by an aspartic acid residue, acts as a IL-13 receptor antagonist.

IL-13 down-modulates macrophage activity, reducing the production of pro-inflammatory cytokines (IL-1, IL-6, IL-8, IL-10, IL-12) and chemokines in response to IFN-gamma or bacterial lypopolysaccharides. IL-13 decreases the production of nitric oxide by activated macrophages, leading to a decrease in parasiticidal activity. IL13 induces human monocyte differentiation, enhances survival time in culture, and also induces B-cell differentiation and proliferation and isotype switching. It induces IL-4 independent IgG4 and IgE synthesis in human B-cells and germ-line IgE heavy chain gene transcription. IL-13, like IL-4, induces CD23 expression on B-cells and enhances CD72, and class II major histocompatibility complex antigen expression. IL-13 increases the killer activity of LAK cells (lymphokine-activated killer cells ) induced by IL-2.

IL-13 can be detected by bioassays involving the use of a subclone of the B9 hybridoma cell line. Human and murine IL-13 activities are assayed by employing human TF-1 erythroleukemia cells. Other methods, including flow cytometry and ELISA, can also be used.

IL-13 neutralizing agent: An agent which decreases the biological activity of IL-13, for example to an IL-13 activity level below that which can be detected using a standard immunoassay. Such agents can thus can be used to inhibit IL-13 activity. Examples of such agents, include, but are not limited to anti-IL-13 antibodies and soluble IL-13 receptor. Particular examples include monoclonal IL-13 antibodies. In a particular example, an IL-13 neutralizing agent is an IL-4/IL-13 Trap (Regeneron, Tarrytown, N.Y.) that binds to and neutralizes both IL-4 and IL-13.

Isolated: An “isolated” biological component (such as a nucleic acid molecule, protein or portion of hematological material, such as blood components) has been substantially separated or purified away from other biological components in the cell of the organism in which the component naturally occurs. Nucleic acids and proteins that have been “isolated” include nucleic acids and proteins purified by standard purification methods. The term also embraces nucleic acids and proteins prepared by recombinant expression in a host cell, as well as chemically synthesized nucleic acids and proteins.

An isolated cell is one which has been substantially separated or purified away from other biological components of the organism in which the cell naturally occurs. For example, an isolated CD4⁺ cell population is a population of CD4⁺ cells which is substantially separated or purified away from other blood cells, such as CD8⁺ cells. An isolated Th1 cell population is a population of Th1 cells which is substantially separated or purified away from other blood cells, such as Th2 cells.

Lymphocytes: A type of white blood cell involved in the immune defenses of the body. There are two main types of lymphocytes: B-cells and T-cells.

Lymphoproliferation: An increase in the production of lymphocytes.

Malignant: Cells which have the properties of anaplasia invasion and metastasis.

Mammal: Includes both human and non-human mammals. Examples of mammals include, but are not limited to: primates (such as apes and chimpanzees), dogs, cats, rats, mice, cows, pigs, sheep, horses, goats, and rabbits.

Monocyte: A large white blood cell in the blood that ingests microbes or other cells and foreign particles. When a monocyte passes out of the bloodstream and enters tissues, it develops into a macrophage.

Neoplasm: Abnormal growth of cells.

Neutralizing amount: An amount of an agent sufficient to decrease the activity or amount of a substance to a level that is undetectable using standard methods.

Non-cultured Cells: Cells which have not been grown or expanded outside of the body. In one embodiment, non-cultured CD4⁺ and CD8⁺ T cells are cells that have been removed and purified from the body, but not grown in culture.

Normal Cell: Non-tumor cell, non-malignant, uninfected cell.

Purified: The term “purified” does not require absolute purity; rather, it is intended as a relative term. Thus, for example, a substantially purified protein, nucleic acid, or cell is one in which the protein, nucleic acid, or cell is more pure than the protein, nucleic acid, or cell in its natural environment, such as within a cell or within an organism. In particular examples, substantially purified populations of cells refers to populations of cells that are at least 70%, 75%, 80%, 90%, 95%, 96%, 97%, 98% or 99% pure. In one embodiment, a substantially purified population of Th1 cells is composed of about 95% Th1 cells, that is the population of cells includes less than about 5% of other T lymphocytes such as Th2 cells. The purity of a Th1 population can be measured based on cell surface characteristics (e.g. as measured by fluorescence activated cell sorting) or by cytokine secretion profile (e.g. as measured by an ELISA assay), as compared to a control.

Thus, in one embodiment, a population of substantially purified CD4⁺ T cells demonstrates a 95% reduction in IL-4 secretion relative to a control Th2 population from the same donor. In another embodiment, a population of substantially purified Th1 cells is about 99% Th1 cells, that is the population of cells includes less than about 1% of other T lymphocytes such as Th2 cells. In one specific, non-limiting example, a substantially purified population of CD4⁺ T cells demonstrates a 99% reduction in IL-4 secretion relative to a control CD4⁺Th2 population from the same donor.

One specific, non-limiting example of a substantially purified population of CD4⁺ Th1 cells is a CD4⁺ population of cells that produces less than 200 pg/ml of IL-4 per 1×10⁶ CD4⁺ Th1 lymphocytes, for example less than 100 pg/ml of IL-4 per 1×10⁶ CD4⁺ Th1 lymphocytes, for example less than 10 pg/ml of IL-4 per 1×10⁶ CD4⁺ Th1 lymphocytes. In further embodiments, a substantially purified population of Th1 cells is a CD4+ population of cells that produces at least 200 pg/ml of IL-2 per 1×10⁶ CD4⁺ Th1 lymphocytes, for example at least 500 pg/ml of IL-2 per 1×10⁶ CD4⁺ Th1 lymphocytes, for example at least 1000 pg/ml of IL-2 per 1×10⁶ CD4⁺ Th1 lymphocytes.

Reconstituting immunity: Increasing the number of lymphocytes, for example increasing the number of lymphocytes in an immuno-depleted subject, such that the immune system of the subject is enhanced relative to the immune system during immuno-depletion.

Stem Cell: A pluripotent cell that gives rise to progeny in all defined hematolymphoid lineages. In addition, limiting numbers of cells are capable of fully reconstituting a seriously immunocompromised subject in all blood cell types and their progenitors, including the pluripotent hematopoietic stem cell by cell renewal.

Subject: Includes any organism having a vascular system and hematopoietic cells in the wild-type organism. In one embodiment, the subject is a mammalian subject, such as a human or veterinary subject.

Substantially Free: Below the limit of detection for a given assay. Thus, in one specific non-limiting example, a cell culture is substantially free of IL-4 if it cannot be detected by a standard assay for analyzing IL-4 expression (e.g. below 10 pg/ml IL-4). In one embodiment, the assay is a bioassay or an ELISA assay for a specific cytokine, wherein appropriate controls are utilized to document the absence of expression of the cytokine.

Supernatant: The culture medium in which a cell is grown. The culture medium includes material from the cell, including secreted growth factors.

Therapeutically Effective Amount: An amount sufficient to achieve a desired biological effect, for example an amount that is effective to increase an immune response. In particular examples, it is an amount of Th1 cells effective to increase an immune response, such as in a subject to whom it is administered, such as a subject having cancer or having at least one infectious disease. In other examples, it is an amount effective to increase an immune response by more than a desired amount, for example by at least 10%, 20%, or even 50%.

In one embodiment, the therapeutically effective amount also includes a quantity of purified Th1 cells sufficient to achieve a desired effect in a subject being treated. For instance, these can be an amount necessary to improve signs and/or symptoms a disease such as cancer or an infection, for example by increasing an immune response.

An effective amount of purified Th1 cells can be administered in a single dose, or in several doses, for example daily, during a course of treatment. However, the effective amount of purified Th1 cells will be dependent on the subject being treated, the severity and type of the condition being treated, and the manner of administration. For example, a therapeutically effective amount of purified Th1 cells can vary from about 5×10⁶ cells per kg body weight to about 1.25×10⁸ cells per kg body weight.

The methods disclosed herein have equal application in medical and veterinary settings. Therefore, the general term “subject being treated” is understood to include all organisms (e.g. humans, apes, dogs, cats, horses, and cows) that require an increase in the desired biological effect, such as an enhanced immune response.

Therapeutically effective dose: A dose of purified Th1 cells sufficient to increase an immune response in a subject to whom it is administered, resulting in a regression of a pathological condition, or which is capable of relieving signs or symptoms caused by the condition. In a particular embodiment, it is a dose of purified Th1 cells sufficient to increase an anti-tumor immune response. In yet another embodiment, it is a dose of purified Th1 cells sufficient to improve a subject's response to an infection. In another embodiment, it is a dose sufficient to enhance vaccine therapy.

T Cell: A white blood cell critical to the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, CD8 T cells are cytotoxic T lymphocytes. In another embodiment, a CD8 cell is a suppressor T cell.

T cell stimulation: A state in which a T cell response has been initiated or activated by a primary signal, such as through the TCR/CD3 complex, but not necessarily due to interaction with a protein antigen. T cell stimulation includes stimulation of a T cell with a primary signal (e.g. anti-CD3) and a co-stimulatory molecule (e.g. anti-CD28). A T cell is activated if it has received a primary signaling event that initiates an immune response by the T cell.

T cell stimulation can be accomplished, for example, by stimulating the T cell TCR/CD3 complex or via stimulation of the CD2 surface protein. An anti-CD3 monoclonal antibody can be used to activate a population of T cells via the TCR/CD3 complex. A number of anti-human CD3 monoclonal antibodies are commercially available. For example, OKT3 prepared from hybridoma cells obtained from the American Type Culture Collection (ATCC, Manassas, Va.) and monoclonal antibody G19-4 can be used to activate T cells. Similarly, binding of an anti-CD2 antibody will activate T cells.

Th1 and Th2 Cells: Type-1 helper cells (Th1), but not type-2 helper cells (Th2), are CD4⁺ T cells that secrete Th1 cytokines. Specific, non-limiting examples of Th1 cytokines are IL-2, IL-12, interferon gamma (IFN-γ), tumor necrosis factor beta (TNF-β), and in some embodiments, IL-13. Th2 cells, but not Th1 cells, express Th2 cytokines. Specific, non-limiting examples of Th2 cytokines are IL-4, IL-5, IL-6, and in some embodiments, IL-10.

The different patterns of cytokine secretion have been postulated correspond with different functions as immune effectors. Th1 cells promote cell-mediated effector responses, while Th2 cells are helper cells that influence B-cell development and augment humoral responses such as the secretion of antibodies, predominantly of IgE, by B-cells. Both types of Th cells influence each other by the cytokines they secrete. For example, IFN-γ inhibits proliferation of murine Th2 cells but not Th1 helper T-lymphocyte clones. In contrast, IL-10 from Th2 cells can inhibit the proliferation of Th1 cells. This Th1/Th2 cell cross-regulation has been demonstrated both in vitro and in vivo. That is, multiple murine models, including infectious disease, cancer, transplantation, and autoimmune models, have demonstrated that such a Th1/Th2 immune balance contributes significantly to the natural history of these various conditions.

Tumor: A neoplasm. Includes solid and hematological (or liquid) tumors.

Examples of hematological tumors include, but are not limited to: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrdm's macroglobulinemia, heavy chain disease, myelodysplastic syndrome, and myelodysplasia.

Examples of solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Tumor necrosis factor beta (TNF-β): Includes both naturally occurring TNF-β, peptides, as well as TNF-β fragments and variants that retain full or partial TNF-β biological activity. The human TNF-β protein is 171 amino acids and is N-glycosylated at position 62. Murine and human TNF-β are highly homologous (74%). Recombinant human proteins with deletions of 27 amino acids from the N terminus are biologically active in several bioassays.

The TNF-β gene has a length of approximately 3 kb, contains four exons, and maps to human chromosome 6p23-6q12. It encodes a primary transcript of 2038 nucleotides yielding a mRNA of 1.4 kb. The 5′ region of the TNF-beta promoter contains a poly(dA-dT)-rich sequence that binds the non-histone protein HMG-I which is involved in the regulation of the constitutive expression of the gene.

TNF-β is produced predominantly by mitogen-stimulated T-lymphocytes and leukocytes. The factor is secreted also by fibroblasts, astrocytes, myeloma cells, endothelial cells, epithelial cells and a number of transformed cell lines. The synthesis of TNF-β is stimulated by interferons and IL-2. TNF-β acts on a plethora of different cells. This activity is not species-specific. Human TNF-beta acts on murine cells but shows a slightly reduced specific activity.

TNF-β can be detected in bioassays involving cell lines that respond to it (such as BT-20, KYM-1D4, L929, L-M, WEHI-3B). TNF-β can also be detected by ELISA and an immunoradiometric assay (IRMA). An alternative detection method is RT-PCR quantitation of cytokines.

Transplantation: The transfer of a tissue, cells, or an organ, or a portion thereof, from one subject to another subject, from one subject to another part of the same subject, or from one subject to the same part of the same subject. In one embodiment, transplantation of CD4⁺ cells, such as a substantially purified population of Th1 cells, into the same subject involves removal of blood from the subject, purification and generation of Th1 cells ex vivo, and introduction of the substantially purified Th1 cells into the same subject.

An allogeneic transplant or a heterologous transplant is transplantation from one individual to another, wherein the individuals have genes at one or more loci that are not identical in sequence in the two individuals. An allogeneic transplant can occur between two individuals of the same species, who differ genetically, or between individuals of two different species. An autologous transplant is transplantation of a tissue, cells, or a portion thereof from one location to another in the same individual, or transplantation of a tissue or a portion thereof from one individual to another, wherein the two individuals are genetically identical.

Vaccine: A composition containing at least one antigen, such as: a live but weakened virus, an inactivated bacteria, virus, or toxoid (or portions thereof, such as a protein); or tumor antigen. Administration of a vaccine causes the body to produce antibodies against the antigen, which in one embodiment prevents the subject from getting the disease which the virus, toxin, or bacterium causes. In another embodiment, a vaccine enhances a subject's immune system to treat a tumor in the subject. The antibodies produced as a result of the vaccine prevent future illnesses of the disease, and thus provide immunity to the subject. Examples of common vaccines include, but are not limited to, those for: hepatitis B, measles, mumps, rubella, polio, influenza, tetanus, diphtheria, as well as anti-tumor vaccines. Vaccines can be administered orally or by injection.

### Method for Purifying and Expanding CD4⁺ Th1 Cells

A method of producing a population of substantially purified CD4⁺ Th1 lymphocytes is provided herein. The method includes isolating or obtaining CD4⁺ cells from a subject. In one example, the method includes further purifying a CD4⁺RA⁺ T cell subset of CD4⁺ cells. In one example the subject has at least one infectious disease, such as a bacterial, viral, parasitic, or fungal infection. In another embodiment, the subject has at least one tumor, such as a solid or hematological tumor.

In one example, CD4⁺ T cells are isolated via cell sorting. One specific, non-limiting example of a method of isolating CD4⁺ cells is the use of negative magnetic immunoadherence. This method uses a cocktail of monoclonal antibodies directed to cell surface markers present on the cells negatively selected. For example, to isolate cells, a monoclonal antibody cocktail may include antibodies to CD14 (e.g. monoclonal antibody 63D3, or 20.3), CD20 (e.g. monoclonal antibody IF5 or Leu-16), CD11b (monoclonal antibody OKMI or 60.1), CD16 (monoclonal antibody FC-2.2 or 3G8), HLA-DR (e.g. monoclonal antibody 20.6 or HB10a and CD8 (e.g. monoclonal antibody OKT8, 51.1, or G10-1.1). This process of negative selection results in an essentially homogenous population of CD4⁺ cells (see U.S. Pat. No. 5,858,358). However, this method is exemplary, other methods known to those of skill in the art can also be utilized.

In another example, purified populations of CD4⁺RA⁺ T cells are isolated via cell sorting. One specific, non-limiting example of a method of isolating CD4⁺RA⁺ T cells is the use of positive selection. Using antibodies directed to the RA antigen on CD4 cells to mark the RA subset of CD4 cells, the CD4⁺RA⁺ T cells can be purified by flow sorting.

The purified CD4⁺ T cells are stimulated by contacting the cells with an anti-CD3 antibody and antibody that specifically binds to a T cell costimulatory molecule. In one embodiment, the antibodies are immobilized. In a particular embodiment, the antibodies are immobilized on a bead, a magnetic solid phase surface, or adhered to a tissue culture flask. T cell costimulatory molecules include, but are not limited to: CD28; inducible costimulatory molecule (ICOS); 4-1BB receptor (CDw137); lymphocyte function-associated antigen-1 (LFA-1); CD30; and CD154. Methods of stimulation of T cells with immobilized anti-CD3 and an immobilized costimulatory molecule are known (see U.S. Pat. No. 3,858,350 and PCT WO 94/29436, herein incorporated by reference in their entirety). The CD4⁺ T cells can be stimulated once. In an alternative embodiment, the population of T cells is re-stimulated by contacting the cells with an immobilized anti-CD3 antibody and an immobilized antibody that specifically binds to a T cell costimulatory molecule. For example the re-stimulation of the T-cells can occur within about eight to about twelve days of the initial stimulation of the T cells.

Stimulation of the CD4⁺ T cells is performed in the presence of a Th1 supportive environment, and the cells are allowed to proliferate in the Th1 supportive environment. In one embodiment, the Th1 supportive environment comprises at least 20 IU/ml of IL-2, for example at least 50 IU/ml of IL-2, for example at least 100 IU/ml of IL-2, for example at least 200 IU/ml of IL-2, for example at least 300 IU/ml of IL-2, for example at least 400 IU/ml of IL-2, for example at least 500 IU/ml of IL-2, for example at least 750 IU/ml of IL-2, for example at least 1000 IU/ml of IL-2, and a neutralizing amount of an IL-4 neutralizing agent. Examples of IL-4 neutralizing agents that can be used to practice the methods disclosed herein, include, but are not limited to: anti-IL-4 antibodies, such as anti-IL-4 monoclonal antibodies and an IL-4/IL-13 trap (Regeneron); and soluble IL-4 receptors.

In some examples, Th1 supportive environment further includes an IL-13 neutralizing agent and/or an agent that neutralizes both IL-4 and IL-13, such as an IL-4/IL-13 trap (Regeneron). In another example, the Th1 supportive environment further comprises at least 1 ng/ml of IL-12, for example at least 2.5 ng/ml, such as about 2.5 ng/ml of IL-12, for example at least 10 ng/ml IL-12, for example at least or about 20 ng/ml IL-12. In another embodiment, the Th1 supportive environment comprises about 1000 IU/ml of IL-2 and a neutralizing amount of an IL-4 neutralizing agent. In some embodiments, the Th1 supportive environment comprises about 1000 IU/ml of IL-2 and a neutralizing amount of an IL-4 and an IL-13 neutralizing agent, such as an IL-4/IL-13 trap. Examples of particular amounts of IL-4/IL-13 trap to add include, but are not limited to about 0.1-1 μg/ml, such as less than about 1 μg/ml. Other examples of particular amounts of IL-4/IL-13 trap to add include about 10⁻¹⁰-10⁻⁹ M. In another embodiment, the Th1 supportive environment further comprises at least 1 ng/ml of IL-12, for example at least 2.5 ng/ml, such as about 2.5 ng/ml of IL-12, for example at least 10 ng/ml IL-12, for example at least or about 20 ng/ml IL-12. In one particular example, the Th1 supportive environment comprises about 1000 IU/ml of IL-2, a neutralizing amount of an IL-4 and an IL-13 neutralizing agent, and at least 1 ng/ml of IL-12.

In one embodiment, the substantially purified CD4⁺ Th1 lymphocytes secrete a Th1 cytokine. In another embodiment, the substantially purified CD4⁺ Th1 lymphocytes are substantially free of secretion of a type II cytokine. For example, the Th1 lymphocytes do not secrete measurable amounts of IL-4 but do secrete measurable amounts of IL-2. In a particular embodiment, the Th1 cells secrete IL-2 and/or INF-γ but not measurable or detectable amounts IL-4. In yet another embodiment, the Th1 cells do not secrete detectable amounts of IL-10. In a particular example, purified CD4⁺ Th1 cells produce less than 10 pg/ml of IL-4 per 1×10⁶ CD4⁺ Th1 lymphocytes. In yet another example, Th1 lymphocytes produce at least 1000 pg/ml of IL-2 per 1×10⁶ CD4⁺ Th1 lymphocytes. The secretion of cytokines can be measured using standard bioassays, such as an ELISA.

In one embodiment, the population of substantially purified cells produced has less than 5% Th2 lymphocytes, or less than 1% Th2 lymphocytes. The proportion of Th2 lymphocytes in the population can be measured by any means known to one of skill in the art. For example, fluorescence activated cell sorting can be utilized. Alternatively the supernatant content is tested for secretion of cytokines. In one embodiment, an assay, such as a bioassay, and ELISA, or a radioimmuno assay, is performed to test the cytokine secretion profile of the cells.

The methods disclosed herein can further comprise cryo-preserving the purified CD4⁺ Th1 lymphocytes.

Also comprehended by this disclosure are CD4⁺ Th1 cells produced by the method disclosed herein. In one embodiment, a substantially purified population of CD4⁺ Th1 lymphocytes has less than 5% CD4⁺ Th2 lymphocytes, such as less than 1% CD4⁺ Th2 lymphocytes. In another embodiment, the substantially purified population of CD4⁺ Th1 lymphocytes produces less than about 10 pg/ml of IL-4 per 1×10⁶ CD4⁺ Th1 lymphocytes. In yet another embodiment, the substantially purified population of CD4⁺ Th1 lymphocytes produces at least 1000 pg/ml of IL-2 per 1×10⁶ CD4⁺ Th1 lymphocytes.

### Methods for Treatment by Transplanting Purified/Expanded Th1 Cells

The ability of a subject to overcome pathological conditions, such as an infectious disease or even a tumor, is reduced if the subject has low levels of Th1 cells. Therefore, by purifying and generating a substantially purified population of Th1 cells from a subject ex vivo and introducing a therapeutic amount of Th1 cells into the same subject, or into another subject (heterologous transplant), the immune system of the subject will be enhanced towards a type I cytokine profile, thus treating the infection or tumor. The Th1 cells can be administered at a dose of about 5×10⁶ to about 2×10⁸ substantially purified CD4⁺ Th1 lymphocytes per kilogram of subject. In addition, substantially purified populations of CD4⁺ Th1 lymphocytes from the subject can be cryopreserved and thawed prior to administration to the subject.

The substantially purified populations of CD4⁺ Th1 lymphocytes disclosed herein can be administered with a pharmaceutically acceptable carrier, such as saline. In one embodiment, compositions containing substantially purified populations of CD4⁺ Th1 lymphocytes can also contain one or more therapeutic agents, such as one or more anti-microbial and/or anti-tumor agents, and/or non-cultured CD4⁺ and CD8⁺ T cells. Other therapeutic agents that can be used to practice the methods disclosed herein include, but are not limited to vaccines, such as an anti-tumor vaccine; immune-depleting agents, such as a chemotherapeutic agent or a monoclonal antibody therapy. Such agents can be administered before, during, or after administration of the Th1 cells, depending on the desired effect. In one example, a population of substantially purified CD4⁺ Th1 lymphocytes from the subject is generated prior to administration of immune-depleting agents, and the Th1 cells administered subsequent to the administration of immune-depleting agents.

Also disclosed herein is a method for enhancing a vaccine response, using substantially purified CD4⁺ Th1 lymphocytes disclosed herein. Administration of substantially purified CD4⁺ Th1 lymphocytes from the subject before, during, or after vaccination enhances the immune response against the antigen(s) present in the vaccine.

In addition, a method of transplanting autologous immune cells to reconstitute immunity in an immuno-depleted subject having a tumor, is provided herein. The method includes immuno-depleting the subject. A therapeutically effective amount of a population of autologous cells including CD4⁺ and CD8⁺ T cells is administered to the subject, as well as a therapeutically effective amount of a population of substantially purified CD4⁺ Th1 lymphocytes (obtained using the methods disclosed herein). In one example, such cells are obtained prior to immuno-depleting the subject. The method results in transplanting autologous immune cells into the subject and reconstituting immunity in the subject. In one example, the Th1 cells activate the CD4⁺ and CD8⁺ cells towards a type I immunity, resulting in enhanced immune-mediated anti-tumor effects. Similar methods can be used to transplant heterologous immune cells, which are not rejected by the recipient.

The subject's immune system, such as T cells, can be non-selectively or selectively depleted, or ablated, by any method known in the art, for example, selective depletion or ablation of T cells or a specific subset of T cells. In one example, the subject's immune system is depleted or ablated by the administration of an induction chemotherapy regimen comprising a therapeutically effective amount of etoposide, doxorubicin, vincristine, cyclophosphamide, and prednisone (EPOCH). In another embodiment, fludarabine is administered to improve the depletion of T cells.

Following depletion or ablation of the immune system, a therapeutically effective amount of a population of autologous cells, including CD4⁺ and CD8⁺ T cells, are administered to the subject. In one example, lymphocytes are collected by apheresis. In one specific non-limiting example, the lymphocyte fraction is collected by elutriation of the lymphocytes and depletion of the B cells. In another embodiment, the lymphocyte fraction is collected by elutriation and enriched for CD34⁺ cells.

Substantially purified CD4⁺ Th1 lymphocytes are prepared by the methods disclosed herein. A therapeutically effective amount of autologous cells including CD4⁺ and CD8⁺ T cells and a therapeutically effective amount of a population of substantially purified CD4⁺ Th1 lymphocytes can be administered to the subject. Specific, non-limiting examples of a therapeutically effective amount of substantially purified CD4⁺ Th1 lymphocytes include substantially purified CD4⁺ Th1 lymphocytes administered at a dose of about 5×10⁶ cells per kilogram to about 125×10⁶ cells per kilogram, or from about 5×10⁶ cells per kilogram to about 25×10⁶ cells per kilogram, or at about 25×10⁶ cells per kilogram, or at about 125×10⁶ cells per kilogram.

The substantially purified CD4⁺ Th1 lymphocytes are administered at the same time, directly following, or at a time remote from the administration of the autologous cells including CD4⁺ and CD8⁺ T cells. In one specific non-limiting example, the substantially purified CD4⁺ Th1 lymphocytes are administered within one day of the autologous cells including CD4⁺ and CD8⁺ T cells. In another specific, non-limiting example, the Th1 cells are administered along with autologous CD4⁺ and CD8⁺ cells that are contained in a peripheral blood stem cell transplant (PBSCT).

In one embodiment, the dose of autologous CD4⁺ and CD8⁺ T cells administered to the subject is from about 40×10⁶ T cells per kg to about 400×10⁶ T cells per kg. In another embodiment, the dose of autologous CD4⁺ and CD8⁺ T cells is included in a peripheral blood stem cell transplant product.

Disclosure of certain specific examples is not meant to exclude other embodiments. In addition, any treatments described in the specification are not necessarily exclusive of other treatment, but can be combined with other bioactive agents or treatment modalities.

### EXAMPLE 1

### Ex Vivo Generation of CD4⁺ Th1 Cells

Lymphocyte Harvest and T Cell Isolation

A subject underwent a 2 to 5 liter apheresis procedure using a CS-3000 or an equivalent machine to collect lymphocytes. The apheresis product was subjected to counterflow centrifugal elutriation using standard methods. ACK lysis buffer (Biofluids, Inc., Rockville, Md.) was used to remove red blood cells from the apheresis product The lymphocyte fraction of the elutriation product (120 to 140 fraction) was depleted of B cells by incubation with a mouse anti-human-B cell antibody (for example anti-CD20; anti-CD22; or anti-CD23; Baxter) and a mouse anti-human-CD8 antibody (Nexell) followed by incubation with sheep anti-mouse magnetic beads (Dynal; obtained through Nexell) by standard methods using the MaxCep Device (Nexell). Cells isolated by this type of procedure have been infused without any toxicity that can be attributed to the selection procedure. Flow cytometry was performed to document that CD8⁺ T cell contamination was <1%. The resultant CD4⁺-enriched lymphocyte product was cryopreserved in aliquots of 50 to 200×10⁶ cells/vial.

Ex vivo Generation of CD4⁺ Th1 Cells

Cryopreserved CD4⁺ T cells were resuspended in filtered flasks to a concentration of 0.3×10⁶ cells per ml, in X-Vivo 20 (BioWhitaker) supplemented with 5% heat-inactivated autologous plasma (herein referred to as “media”). Cells were grown at 37° C. in 5% CO₂ humidified incubators. At the time of culture initiation, T cells were stimulated with anti-CD3/anti-CD28 coated magnetic beads (3 to 1 ratio of beads to T cells). Tosylated magnetic beads (Dynal) are conjugated with an antibody to human CD3 (clone OKT3) and an antibody to human CD28 (clone 9.3). In 50 infusions of T cells grown with anti-CD3/anti-CD28 coated beads, there have been no adverse reactions except the development of an asymptomatic HAMA serologic response in one patient.

At the time of co-culture initiation and on day 2 of culture, the following reagents were added to the media: recombinant human IL-2 (Chiron Therapeutics; 1000 I.U. per ml), 2.5 ng/ml recombinant human IL-12 (R&D Systems, Minneapolis, Minn.; catalog # 219-IL-005), and a neutralizing amount of antibody to IL-4 (American Type Culture Collection (ATCC), Manassas, Va.; ATCC Number HB-9809; clone designation for this cell line is clone MP4.25D2.11). A “neutralizing amount” of an IL-4 neutralizing agent, such as an antibody to IL-4, is an amount required to decrease the level of recombinant human IL-4 to an undetectable level in an ELISA assay as performed using the manufacturer's instructions (IL-4 ELISA available from BioSource International, Camarillo, Calif.). The ELISA was conducted on Th1 cell supernatants after stimulation of cells with anti-CD3, anti-CD28 beads. If the cells have 99% less IL-4 than the level of IL-4 produced by a control Th2 cell culture (i.e. the level of IL-4 is below the 10 pg/ml detection limit for the IL-4 ELISA assay), then the cells are >99% pure for a Th1 profile. For example, a level of IL-4 of less than 10 pg/ml (per million CD4⁺ cells for a 24 hour period of supernatant generation) demonstrates a >99% purity of Th1 cells.

After day 2, cells were maintained at a concentration of 0.25 to 1.0×10⁶ cells per ml by the addition of fresh media supplemented with IL-2 (1000 I.U./ml) and a neutralizing amount of antibody to IL-4 (see above). The median cell volume was determined using a Multisizer II instrument (Coulter). When the T cell volume approached 500 femtoliters (fl) (acceptable range of 650 to 350), the T cells were restimulated with anti-CD3/anti-CD28 beads. Typically, this time of restimulation was after 7 to 10 days of culture. Bead restimulation was at a bead to T cell ratio of 3:1. T cell concentration was 0.2×10⁶ cells/ml. Media again was supplemented with IL-2 (1000 I.U./ml) and a neutralizing amount of antibody to IL-4 (see above).

After bead restimulation, CD4 cells were maintained at a concentration of 0.25 to 1.0×10⁶ cells per ml by the addition of fresh media supplemented with IL-2 (1000 I.U./ml) and a neutralizing amount of antibody to IL-4 (see above). When the CD4 cell mean cell volume approached 500 fl (acceptable range of 650 to 350), the cells were harvested and cryopreserved at 10-100×10⁶ cells/ml using standard methods. Generally, the total time of CD4 cell culture was 15 to 20 days.

### EXAMPLE 2

### Demonstration of Th1 Cell Expansion

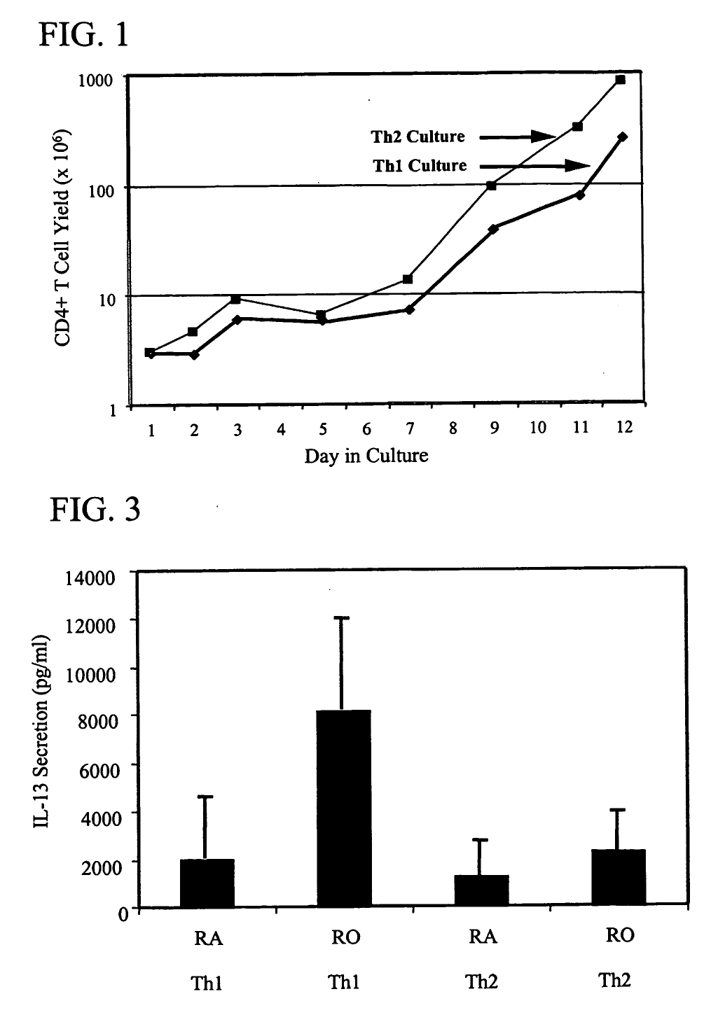

Human CD4⁺ cells were stimulated ex vivo as described in EXAMPLE 1. Briefly, human peripheral blood lymphocytes were collected by apheresis and subsequently purified by counterflow centrifugal elutriation. CD4⁺ T cells were enriched for by negative selection using anti-CD8 and anti-CD20 antibodies and sheep anti-mouse magnetic beads. Two rounds of antibody depletion were performed to ensure that CD8⁺ T cell content was less than 0.5% of the starting T cell population. CD4-enriched T cells were plated in tissue culture flasks at a concentration of 200,000 cells per ml of culture media, comprising X-Vivo 20 media (BioWhitaker) supplemented with 5% autologous plasma. Anti-CD3, anti-CD28 coated magnetic beads were added to the culture at a T cell to bead ratio of 1:3. In the Th1 culture flask, recombinant human IL-2 (1000 I.U./ml), recombinant human IL-12 (2.5 ng/ml), and neutralizing amount of an antibody to IL-4 were added (see above). In the Th2 culture flask, recombinant human IL-2 (20 I.U./ml) and recombinant human IL-4 (1000 I.U./ml) were added. The growth of the cells was evaluated over time. As shown in FIG. 1, CD3/CD28 stimulation resulted in CD4⁺ cell expansion in both the Th2 and the Th1 culture conditions.

### EXAMPLE 3

### Cytokine Secretion Profile of Th1 Cells

Cells were prepared as described above. Briefly, human peripheral blood lymphocytes (PBMCs) were collected by apheresis and subsequently purified by counterflow centrifugal elutriation. CD4⁺ T cells were enriched for by negative selection using anti-CD8 and anti-CD20 antibodies and sheep anti-mouse magnetic beads. Two rounds of antibody depletion were performed to ensure that CD8⁺ T cell content was less than 0.5% of the starting T cell population. CD4-enriched T cells were plated in tissue culture flasks at a concentration of 200,000 cells per ml of culture media, comprising X-Vivo 20 media supplemented with 5% autologous plasma. Anti-CD3, anti-CD28 coated magnetic beads were added to the culture at a T cell to bead ratio of 1:3. In the Th1 culture flask, recombinant human IL-2 (1000 I.U./ml), recombinant human IL-12 (2.5 ng/ml), and neutralizing amount of antibody to IL-4 were added. In the Th2 culture flask, recombinant human IL-2 (20 I.U./ml) and recombinant human IL-4 (1000 I.U./ml) were added.

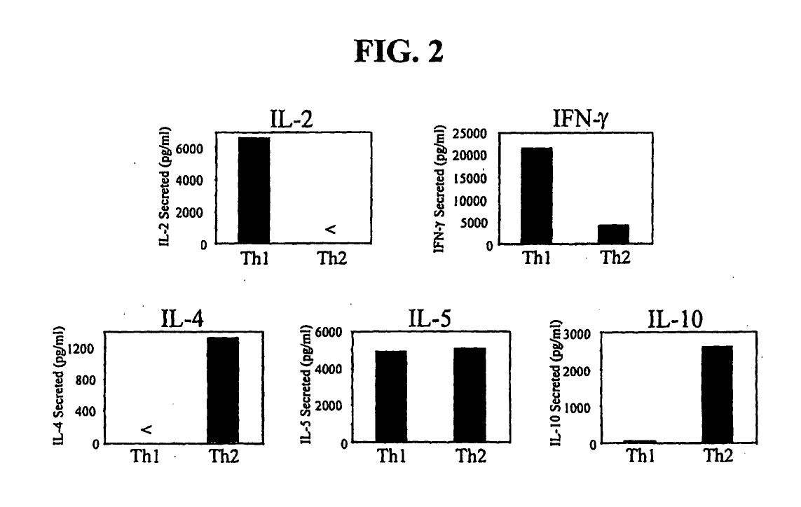

Both Th1 and Th2 cultures were maintained at a concentration of 200,000 cells per ml by the addition of fresh media that was replete with recombinant cytokines. Cultures were monitored for cell volume by Coulter multisizer analysis. When the cell volume approached 650 fl (typically 8 to 12 days in culture), the Th1 and Th2 cells were harvested and restimulated with anti-CD3, anti-CD28 coated beads (1:3 ratio), and further expanded in cytokine-containing media. When the cell volume again returned to approximately 650 fl (typically after an additional 7 days in culture), the cells were restimulated with CD3, CD28-coated beads and a 24 hour supernatant was generated. The Th1 or Th2 supernatant was analyzed for cytokine content by two-site ELISA technique (BioSource). Results are shown in FIG. 2.

As FIG. 2 demonstrates, CD4 cells propagated in the Th1 culture condition produced a high level of the type I cytokines, IL-2 and IFN-γ, upon repeat CD3, CD28 stimulation. In contrast, the CD4 cells propagated in the Th2 culture condition produced an undetectable level of IL-2 and a reduced level of IFN-γ. This demonstrates that the Th1 culture produced a greater level of type I cytokines than the Th2 culture. In comparison, the Th2 culture secreted a high level of the type II cytokine, IL-4, whereas the Th1 culture did not secrete a detectable level of IL-4. Similarly, the Th2 culture produced an increased amount of the type II cytokine IL-10 relative to the Th1 cells.

Therefore, using the Th1 and Th2 culture conditions described herein, CD3/CD28 stimulation of purified human CD4⁺ T cells can be utilized to generate Th1 or Th2 cells. Th1 cells are characterized by their secretion of type I cytokines, such as IL-2 and IFN-γ and their reduced level of secretion of type II cytokines, such as IL-4, IL-5, and/or IL-10. Th2 cells are characterized by their secretion of the type II cytokines such as IL-2 and/or IL-10, and their reduced level of secretion of the type I cytokines, such as IL-2 and/or IFN-γ.

The production of IL-13 by Th1 and Th2 cells was determined as follows. CD4⁺ T cells were isolated from normal donors, and the naive CD45RA⁺ (RA) or memory CD45RO⁺ (RO) subsets further enriched by flow sorting as described in Example 10. The RA or RO cells were stimulated with anti-CD3, anti-CD28 coated beads in Th1 or Th2 culture conditions as described above. The expanded CD4 cells were restimulated on day 10 to 12 of culture with a second round of anti-CD3, anti-CD28 stimulation. After this second round of expansion, typically by day 20 of culture, the CD4 cells, were stimulated a third time with anti-CD3/anti-CD28 and a 24 hour supernatant obtained. These culture superantants were tested for IL-13 content using a two-site ELISA assay (BioSource, Inc.)

Interestingly, CD3, CD28 generated Th1 cells (FIG. 3) secrete significant amounts of the type II-promoting cytokine IL-13. This result is surprising because Th1 cultured cells do not produce significant amounts of the type II promoting cytokine IL-4. Therefore, since IL-13 production from the Th1 cultured cells may reduce the Th1 purity, neutralization of IL-13 in the Th1 culture can further improve Th1 cell purity. Methods to neutralize IL-13 include incubation of cells in an IL-13 neutralizing agent, such as an IL-4/IL-13 trap, using the methods described above for an IL-4 neutralizing agent Such incubation will enhance IL-2 and IFN-γ secretion, and further reduce IL-4, IL-5, and IL-10 production, by Th1 cells.

### EXAMPLE 4

### Treatment of Disease Using Generated Th1 Cells

The Th1 cells of the present disclosure can be used to enhance a subject's immune system towards a type I cytokine profile. Low levels of Th1 cells reduce a subject's ability to fight any type of infectious disease, including, but not limited to bacterial fungal and viral infections. Therefore, administration of Th1 cells to a subject in these clinical settings can improve the subject's immune response to an infection.

Using the methods disclosed above, Th1 cells obtained from the subject are purified and expanded ex vivo. The expanded Th1 cells are introduced at a therapeutically effective dose into the same subject to stimulate the subject's immune system toward a type I cytokine profile.

Lymphocyte Harvest and T Cell Isolation from Subject

Blood is collected from a subject having at least one infectious disease, and a substantially purified population of Th1 cells generated, using the method disclosed in EXAMPLE 1. The subject need not receive any particular treatment prior to harvesting the CD4⁺ cells. Briefly, the subject undergoes a 2 to 5 liter apheresis procedure using a CS-3000 or an equivalent machine. The apheresis product is subjected to counterflow centrifugal elutriation, and the lymphocyte fraction is depleted of B cells. The resultant CD4⁺-enriched lymphocyte product is cryopreserved using standard methods (for example using a combination of Pentastarch and DMSO) in aliquots of 50 to 200×10⁶ cells/vial. Ideally, to qualify for cryopreservation, the cell culture should contain predominately CD4⁺ T cells by flow cytometry (greater than 70% CD4⁺ T cells, and less than 5% contaminating CD8⁺ T cells). Sterility of the population need not be tested at this stage of the Th1 cell generation procedure; such testing can occur after the final co-culture of cells.

Ex vivo Generation of CD4⁺ Th1 Cells

The cryopreserved CD4⁺ T cells are resuspended to a concentration of 0.3×10⁶ cells per ml, and expanded using the method disclosed in EXAMPLE 1. The resulting population of substantially purified Th1 cells can be used immediately, or cryopreserved for future use. For example, the population of substantially purified Th1-cells is at least 80%, 85%, 90%, 95% or even at least 99% pure. If the cells have 99% less IL-4 than the level of IL-4 produced by a control Th2 cell culture (i.e. the level of IL-4 is below the 10 pg/ml detection limit for the IL-4 ELISA assay), then the cells are >99% pure for a Th1 profile. For example, a level of IL-4 of less than 10 pg/ml (per million CD4⁺ cells for a 24 hour period of supernatant generation) demonstrates a >99% purity of Th1 cells.

In addition, if the T cells are tested for fungal and bacterial cultures, using standard testing done on cell products and for endotoxin content, using a limulus assay. Cell products positive for fungal, bacterial, or endotoxin content are discarded. It is noted that T-cells obtained from subjects infected with HIV, will also be infected with HIV, as the virus directly infects CD4⁺ T cells. Therefore, in samples obtained from HIV positive subjects, methods can be used to control HIV infection during CD4 propagation, such as administration of anti-HIV drugs to the culture or gene-transfer approaches.

To estimate the number of Th1 cells that could be obtained from a subject, the following calculations can be used as a guideline. About 0.5×10⁶ CD4⁺ T cells can be harvested from one ml of blood. Assuming a 2-log expansion of Th1 cells in culture, it is estimated that 5×10⁷ Th1 cells could be generated from one ml of blood. This value assumes 100% efficiency at each step of the process, which is likely not to occur; a range of 20-100% efficiency is reasonable. Therefore, about 1-5×10⁷ Th1 cells could be generated per one ml of blood.

Administration of Generated Th1 Cells

On day 1 of the transplant procedure, Th1 cells are administered intravenously. If the Th1 cells were previously cryopreserved, the cells are thawed and diluted in saline solution to a volume of approximately 125 to 250 ml for intravenous infusion. Th1 cells can be administered in at least one pharmaceutically acceptable carrier, such as a saline solution. In addition, the Th1 cells can be administered concurrently (or separately) with other therapeutic agents, such as anti-microbial agents, for example antibiotics, anti-viral agents, and anti-fungal agents. The Th1 cell therapy can be enhanced by administration of an infectious disease vaccine. In addition to administering substantially purified Th1 cells, non-cultured CD4⁺ and CD8⁺ T cells can be administered with the Th1 cells (concurrently or separately), allowing a more complete CD4⁺ and CD8⁺ immune recovery in a CD4⁺ Th1 and a CD8⁺ Tc1 manner.

Examples of subjects who would benefit from such therapy include, but are not limited to those refractory to other modalities of treatment, for example those subjects having an infection which was not treatable by other means to control the infection (such as standard anti-microbial chemotherapies).

In a particular example, the dose of Th1 cells administered to a subject is in the range of: dose #1, about 5×10⁶ Th1 cells/kg; dose #2, about 2.5×10⁷ Th1 cells/kg; dose #3, about 1.25×10⁸ Th1 cells/kg. Ideally, no cortico-steroids are administered in the management of DMSO-related toxicities (chills, muscle aches) that may occur immediately after cellular infusion (diphenhydramine and meperidine are instead administered). The subject is monitored for the presence or absence of any grade 4 or 5 toxicity attributable to the Th1 cells that occurs in the first 14 days post-transplant.

Toxicity is monitored by criteria established by the National Cancer Institute Cancer Therapy and Evaluation Program (NCI-CTEP). Grade 4 toxicity is considered “life-threatening” whereas Grade 5 toxicity is death. Each organ system (GI system, renal system, nervous system, etc.) is graded on the grade 0 (not observed) to grade 5 scale.

If no grade 4 or 5 toxicity attributable to the Th1 cells is observed in an initial three subjects receiving a particular dose of Th1 cells, then it is determined that that dose level has acceptable toxicity, and accrual to a higher dose level commences. For example, if no grade 4 or 5 toxicity attributable to the Th1 cells is observed in an initial three subjects receiving dose #1, then it is determined that dose level #1 has acceptable toxicity, and accrual to dose level #2 commences. If grade 4 or 5 toxicity attributable to the Th1 cells is observed in any of the initial three subjects, then accrual to dose level #1 is expanded to include a total of six patients. If two subjects in six develop a grade IV toxicity related to the Th1 cells, then it is determined that dose level #1 is not acceptable, and further accrual to the study stops at that point. If only one of the six patients experiences such an adverse effect, then it is determined that dose level #1 has acceptable toxicity, and accrual proceeds to dose level #2.

Three subjects are then subjected to Th1 cell dose level #2 (2.5×10⁷ Th2 cells/kg). The same accrual and stopping rules apply to this dose level as those used for dose level #1. As such, either three or six subjects are accrued to dose level #2.

If it is determined that Th1 cell dose level #2 has acceptable toxicity, accrual to the final dose level #3 starts (Th1 cell dose of 1.25×10⁸ cells/kg). Six subjects are evaluated on dose level #3. If more than one subject on dose level #3 develops a grade 4 or 5 toxicity attributable to the Th1 cells, then accrual to dose level #3 stops.

The Th1 cells disclosed herein can be administered to a subject one or more times as necessary for a particular subject. Although one infusion may be sufficient, several infusions can be performed to increase the benefit, as diseases are oftentimes chronic and difficult to treat. If multiple infusions are performed, they can be separated by a period of about four weeks. During such treatment, the patient is monitored, for example by performing tests about once or twice during each 4 week treatment cycle. Tests would include measurement of T cell cytokines, measurement of immune recovery panels such as T cell counts and T cell diversity and competence using methods known to those skilled in the art. In addition, tests that measure disease activity can also be performed to monitor the beneficial effect of the Th1 cells.

### EXAMPLE 5

### Treatment of Tumors Using Generated Th1 Cells

Th1 cells are associated with an enhanced anti-tumor immune response. As such, the administration of Th1 cells can be therapeutic in subjects having at least one type of tumor, such as cancer. Using the methods disclosed in the EXAMPLES above, Th1 cells are purified and generated ex vivo. As disclosed above, Th1 cells can be administered alone or in the presence of a pharmaceutical carrier, and/or with other cells or therapies. For example, substantially purified Th1 cells can be used to treat a subject having a tumor, alone or in combination with another therapy, such as chemotherapy or monoclonal antibody therapies (see EXAMPLE 7), or an anti-tumor vaccine therapy (see EXAMPLE 6).

If the subject is to receive chemotherapy, the Th1 cells are collected and expanded prior to the chemotherapy, then cryopreserved. The substantially purified Th1 cells are administered to the subject after the chemotherapy, to decrease the subject's risk of developing chemotherapy-resistance disease.

If the subject is to receive a cancer vaccine or monoclonal antibody therapy, the expanded and cryopreserved Th1 cells can be administered to the subject prior to, during, or after the vaccine and/or antibody modalities. Administration of Th1 cells, before, concurrently, or after the vaccination enhances reactivity to the tumor antigens, and by secreting the Th1 cytokines, enhances the vaccine therapy. Administration of Th1 cells, before, concurrently, or after administering a monoclonal antibody therapy enhances the therapy by augmenting the cellular arm of the immune system.

### EXAMPLE 6

### Administration of Th1 Cells as an Adjuvant for a Vaccine

The Th1 cells disclosed herein can also be used as an adjuvant for any vaccine therapy. Administration of Th1 cells, in combination with a vaccine therapy, enhances the immune system towards an antigen(s) present in the vaccine.

Using the methods disclosed in the EXAMPLES above, Th1 cells are purified from a subject and generated ex vivo. The substantially purified Th1 cells can be administered using the methods disclosed herein to a subject prior to, concurrently, or after vaccination of the subject.

As disclosed above, Th1 cells can be administered alone or in the presence of a pharmaceutical carrier, and/or with other cells, such as non-cultured CD4⁺ or CD8⁺ cells from the subject.

Th1 cells can be combined with any anti-tumor vaccine, such as a vaccine which includes one or more peptides which are specific for a mutated or over-expressed tumor antigen, or a whole tumor antigen genes or products. Such vaccines can be administered to individuals by intravenous, intralymphatic, or subcutaneous routes. In addition, the vaccine can be administered alone, or in combination with an immune adjuvant such as Freund's adjuvant or autologous dendritic cells.

### EXAMPLE 7

### Immuno-Depleting a Subject Prior to Administration of Th1 Cells

In some subjects, it may be desirable or necessary to deplete an incompetent immune system and then re-build the immune system by administering a generated population of substantially purified Th1 cells using the methods disclosed in the above EXAMPLES. Any immune-depleting methods can be used. Examples include, but are not limited to immune-depleting chemotherapies and monoclonal antibody therapies.

Immune-depleting Chemotherapies

After cell products are harvested from the subject, chemotherapy is administered. Subjects receive at least one cycle of induction chemotherapy, even if their CD4⁺ count is less than 50 cells per μl. Placement of permanent central venous access can be performed. Ideally, steroids are not used as an anti-emetic during this chemotherapy regimen. Examples of immune depleting chemotherapy that can be used to deplete a patient's immune system prior to Th1 cell therapy include the Fludarabine/EPOCH method (Table 1) and the Fludarabine/cyclophosphamide method (fludarabine (25 mg/m² per day IV for 4 consecutive days) combined with cyclophosphamide (600 mg/m² per day IV for 4 days). However, other methods known to those skilled in the art may also be employed.

TABLE 1
Cycle 1 of Induction Chemotherapy

| Drug | Dose | Days |
|---|---|---|
| Fludarabine | 25 mg/m² per day IV | Days 1, 2, 3 |
| | Infusion over 30 minutes, | |
| | Daily for 3 days | |
| Etoposide | 50 mg/m² per day continuous IV | Days 1, 2, 3 |
| | Infusion over 24 hours, Daily for 3 days | |
| Doxorubicin | 10 mg/m² per day continuous IV | Days 1, 2, 3 |
| | Infusion over 24 hours, Daily for 3 days | |
| Vincristine | 0.5 mg/m² per day continuous IV | Days 1, 2, 3 |
| | Infusion over 24 hours, Daily for 3 days | |
| Cyclophosphamide | 600 mg/m² IV Infusion over 2 hr | Day 4 |
| Prednisone | 60 mg/m² per day orally, daily for 4 days | Days 1, 2, 3, 4 |
| Filgrastim | 10 ug/kg per day subcutaneously | Daily from day 5 Until |
| | | ANC > 1000/μl for two |
| | | consecutive days |

Because the primary purpose of the induction chemotherapy is to establish severe host immune T cell depletion prior to the administration of substantially purified Th1 cells (and/or additional agents such as purified, but uncultured CD4⁺ and CD8⁺ cells), the number of induction chemotherapy cycles administered is determined by the severity of immune T cell depletion observed. The CD4⁺ count can be measured by flow cytometry, for example in the interval of day 15 to day 21 of the fludarabine/EPOCH chemotherapy. If there are >50 CD4⁺ cells per μl of blood during this interval, further cycles of induction chemotherapy are administered (in an attempt to achieve greater immunosuppression prior to transplantation). If there the level of CD4⁺ cells is <50 cells per μl of blood, this indicates that the immune system of the subject is adequately depleted, and that subject receives the transplant preparative regimen.

Subjects receive the second cycle of chemotherapy on day 22 after the first cycle was initiated. However, an additional two weeks of recovery time before administration of the second cycle is provided if medically indicated (for example, for delay in neutrophil recovery, documented infection, or other complication resulting from the induction chemotherapy regimen).

If a subject develops neutropenia of less than 500 PMN's per μl for more than seven days during any cycle of induction chemotherapy, the subject receives no further induction chemotherapy. Instead, they receive a transplant preparative regimen (see below), even if the CD4⁺ count is not <50 cells per μl.

Following chemotherapy, subjects proceed to the transplant preparative regimen chemotherapy (even if the CD4⁺ count is still >50 cells per μl). If a subject develops progressive disease at any point during induction chemotherapy cycles, such a subject proceeds to the transplant preparative regimen (independent of the CD4⁺ count).

Determination of Cycle 2 and Cycle 3 Dose Escalation

If the first cycle of induction chemotherapy does not reduce the CD4⁺ count to below 50 cells per μl and does not result in febrile neutropenia or prolonged neutropenia as evidenced by two consecutive bi-weekly ANC values less than 500 cells per μl, then the next cycle of induction chemotherapy can be dose escalated, by increasing the daily dose of fludarabine, etoposide, adriamycin, and cyclophosphamide 20%. If a third cycle of chemotherapy is required (CD4⁺ count still greater than 50) and febrile neutropenia or two timepoints of ANC less than 500 did not occur after cycle 2, then the third cycle of induction chemotherapy is administered at a further 20% escalation of doses administered for cycle 2.

Dose Reduction of Pre-transplant Induction Chemotherapy

In the event that more than one subject experiences a period of neutropenia (ANC less than 500 per μl) for more than 10 days, the etoposide, doxorubicin, vincristine, and prednisone is reduced from three days to two days of administration. The doses of these medications remain unchanged. In the event of this change, the cyclophosphamide and filgrastim is given on day 3. The same schedule modification described in subsection a) (above) is performed if any grade IV toxicity by the NCI Common Toxicity Criteria is observed in more than one subject.

Transplant Preparative Regimen

On day 22 after the final cycle of induction chemotherapy, subjects are eligible to receive a transplant preparative regimen (see Table 2). Therefore, day 22 of the final induction chemotherapy cycle is transplant day-6. However, in cases where additional recovery time is required (for example, due to prolonged neutropenia, documented infection, or other medical complications of the induction regimen), an additional two weeks of recovery time is utilized prior to initiation of the transplant preparative regimen.

TABLE 2
Transplant Preparative Regimen

| Drug | Dose | Days |
|---|---|---|
| Fludarabine | 30 mg/m² per day IV Infusion | Transplant Days −6, −5, −4, −3 |
| | over 15 to 30 minutes, daily for 4 days | |
| Cyclophosphamide | 1200 mg/m² per day IV Infusion | Transplant Days −6, −5, −4, −3 |
| | over 2 hours, daily for 4 days | |
| Mesna | 1200 mg/m² per day by continuous | Transplant Days −6, −5, −4, −3 |
| | IV Infusion, daily for 4 days | |
| | (start 1 hr before cyclophosphamide)* | |

*Bag #1 of the mesna is 150 mg/m² in 250 ml over a 3 hr infusion (thus stopping when cyclophosphamide ends). Then, mesna is given at 1200 mg/m² in 500 ml over 24 hour infusion, for four days (days −6, −5, −4, and −3).

Hydration Regimen During Preparative Regimen Chemotherapy

Hydration is initiated 12 hours prior to cyclophosphamide infusion (on day-7 of the transplant). Hydration is with normal saline supplemented with 10 meq/liter KCl at a rate of 100 ml/hour. Hydration continues until 24 hours after the last cyclophoshamide dose has been completed. During hydration, 20 mg of furosemide is administered daily by IV route to maintain diuresis. If body weight in any patient increases to more than 5% above pre-cyclophosphamide weight, additional doses of furosemide are administered. In general, furosemide doses are separated by at least a four hour observation interval. During hydration, serum potassium level are monitored every 12 hours. If potassium value is >4.5 meq/l , KCl is removed from the saline infusion. If potassium value is <3.0, KCl concentration in the saline is increased to 25 meq/l. During hydration, if urine output is <1.5 ml/kg/hour, an additional 20 mg of furosemide is administered.

Monoclonal Antibody Therapies

Examples of monoclonal antibody therapies that can be used to practice the disclosed methods include, but are not limited to: Rituxan and Herceptin. Rituxan is a monoclonal antibody to CD20, which is present on B cell malignancies such as lymphoma. Herceptin is a monoclonal antibody to her2-neu, which is often over-expressed on breast cancer cells. These agents are typically administered in combination with chemotherapy. In general, monoclonal-antibody based therapy is well-tolerated so a high degree of monitoring is not required.

### EXAMPLE 8

### Infection Prophylaxis

To assist in protecting a subject from infections that can result from receiving chemotherapy or other immune-depleting therapy, one or more prophylactic compounds can be administered prior to the start of the therapy, to enhance the immune system. The prophylaxis disclosed below may be administered separately, or in combination, depending on the requirements of the subject. In addition, the dosage regimens for the prophylaxis described below are known to those skilled in the art, and can be found in Mandell (Principles and Practice of Infectious Disease; 5th Edition, Copyright 2000 by Churchill Livingstone, Inc.)

For example, at the initiation of pre-transplant induction chemotherapy until administration of immunosuppressive agents is terminated, subjects may receive: trimethoprim 160 mg/sulfamethoxazole 800 mg for PCP prophylaxis (if a subject is allergic to sulfonamide antibiotics, aerosolized pentamadine (300 mg) is administered); fluconazole (oral or i.v.) for fungal and bacterial prophylaxis, and acyclovir for HSV prophylaxis.

### EXAMPLE 9

### Administration of Th1 Cells to Subjects Undergoing Autologous Stem Cell Transplantation

For cancer patients, the development of malignant disease relapse after a stem cell transplant is a very poor prognostic sign. To decrease the incidence of relapse after transplantation, the administration of additional immune cells, such as Th1 cells at the time of relapse can result in tumor regressions. The disclosed Th1 cells can be administered to subjects receiving an autologous stem cell transplant (SCT), to treat the subject and/or as a means of prophylaxis.

Lymphocytes are collected from the subject, and the Th1 cells purified, amplified, and cryopreserved until the subject has received an autologous SCT. The cancer patient is subjected to an immune-depleting therapy (see EXAMPLE 7) to eliminate an immune system that is not efficient in eliminating the cancer. In one embodiment, such immune-depleting chemotherapy includes fludarabine followed by EPOCH chemotherapy, with subsequent administration of fludarabine and higher doses of cyclophosphamide. After immune depletion, the patient receives an autologous SCT (containing CD4⁺ and CD8⁺ T cells in the dose range of 40 to 400×10⁶ T cells per kg). Within 24 hours after this T cell administration, the patient is administered ex vivo generated CD4⁺ Th1 cells, using the methods disclosed herein. This method results in increased type I immunity and enhancement of anti-tumor efficacy.

Peripheral Blood Stem Cell (PBSC) Harvest

Immediately following lymphocyte harvest, the subject receives filgrastim as an outpatient (10 ug/kg/day each morning; subcutaneously) for 5, 6, or 7 days. The subject takes the filgrastim as early as possible upon awakening in the morning. This is especially important on days 5, 6, and 7 of the injections.

Apheresis is typically performed on days 5 and 6. On some occasions, sufficient numbers of CD34⁺ cells can be obtained with a single apheresis on day 5; on other occasions, apheresis is performed on days 5, 6, and 7 to reach the target CD34⁺ cell number (≧4×10⁶ per kg). The subject is instructed to take filgrastim for the complete 7 day period, unless notified by the transplant team that adequate CD34⁺ cells were harvested before day 7.

If ≧3×10⁶ CD34⁺ cells per kg are harvested after apheresis on days 5, 6, and 7, no further mobilization or apheresis is performed, and the patient is eligible to receive the stem cell transplant with that dose of CD34⁺ cells.

In the event that less than 3×10⁶ CD34⁺ cells per kg are harvested after apheresis on days 5, 6, and 7, the subject is given two weeks of rest, and then re-treated with filgrastim followed by repeat peripheral blood stem cell harvesting.

A 15 to 25 liter large volume whole blood apheresis is performed via a 2-armed approach or via a temporary central venous catheter in the femoral position using the Baxter CS3000Plus, Cobe Spectra, or an equivalent instrument. This procedure typically takes 4 to 6 hours.

Apheresis procedure uses ACD-A anti-coagulant; alternatively, partial anti-coagulation with heparin is utilized. The apheresis product is cryopreserved and stored at −180° C. in a solution containing Plasmalyte A, Pentastarch, human serum albumin, DMSO, and preservative free heparin (10 U/ml). The concentration of CD34⁺ cells in the apheresis product is determined by flow cytometry, and the number of CD34⁺ cells in each cryopreserved bag is calculated.

Immunodepletion of the Subject

Following harvest of the lymphocytes, the subject is immuno-depleted using the methods disclosed in EXAMPLE 7.

Transplant Procedure: Autologous Peripheral Blood Stem Cell Transplantation

On day 0, the subject receives the cryopreserved autologous PBSC. The cryopreserved PBSC product is thawed and administered intravenously immediately. The target dose of the PBSC is ≧4×10⁶ CD34⁺ cells per kg. However, if apheresis on days 5, 6, and 7 yielded a total of ≧3×10⁶ CD34⁺ cells per kg, this level of CD34⁺ cell dose is utilized. Ideally, no cortico-steroids are administered in the management of DMSO-related toxicities (chills, muscle aches) that may occur immediately after cellular infusion (diphenhydramine and meperidine are allowed).

On day 0 of the transplant, immediately after PBSC transfusion, patients begin treatment with recombinant human filgrastim at a dose of 10 ug/kg/day s.c. Filgrastim administration continues until the ANC count is greater than 5000 cells per μl for three consecutive days.

Administration of Th1 Cells Post-transplant

Following the transplant, substantially purified Th1 lymphocytes may be administered prophylactically, using the methods disclosed herein, to prevent the recurrence of cancer post-transplant, or administered at any initial sign of cancer recurrence.

### EXAMPLE 10

### Purification of the CD4⁺RA⁺ Subset of CD4⁺ Cells Enhances Th1 Cell Generation

Purified CD4⁺ T cells obtained using the methods disclosed above were further purified into the CD4⁺RA⁺ T cell subset (naive subset) or the CD4⁺RO⁺ T cell subset (memory-type subset). This extra purification step was performed using a positive selection method in which monoclonal antibodies specific for the RA and RO antigens on CD4 cells (PharMingen, Inc.; CD45RA antibody catalog #555488 and CD45RO antibody catalog #555492) were used. After marking the RA and RO subsets of CD4 cells, each population was subsequently purified by flow sorting using a FACSort machine (Becton Dickinson Immunocytometry Systems).

Purified CD4⁺RA⁺ and CD4⁺RO⁺ subsets of CD4 cells were subjected to the Th1 and Th2 culture conditions as detailed in the above examples. Briefly, the RA and RO cells were cultured separately in the Th1 stimulating environment (CD3, CD28 stimulation in the presence of 1000 IU/ml of IL-2, 2.5 ng/ml of IL-12, and the anti-IL-4 monoclonal antibody), or the Th2 stimulating environment (CD3, CD28 stimulation in the presence of 1000 IU/ml of IL-4 and 20 IU/ml of IL-2). After 10 days in culture, each of the four cultures were harvested and re-stimulated with CD3, CD28 beads (1:3 ratio of T cells to beads). A 24 hour supernatant was generated, and tested for cytokine content by two-site ELISA (BioSource).

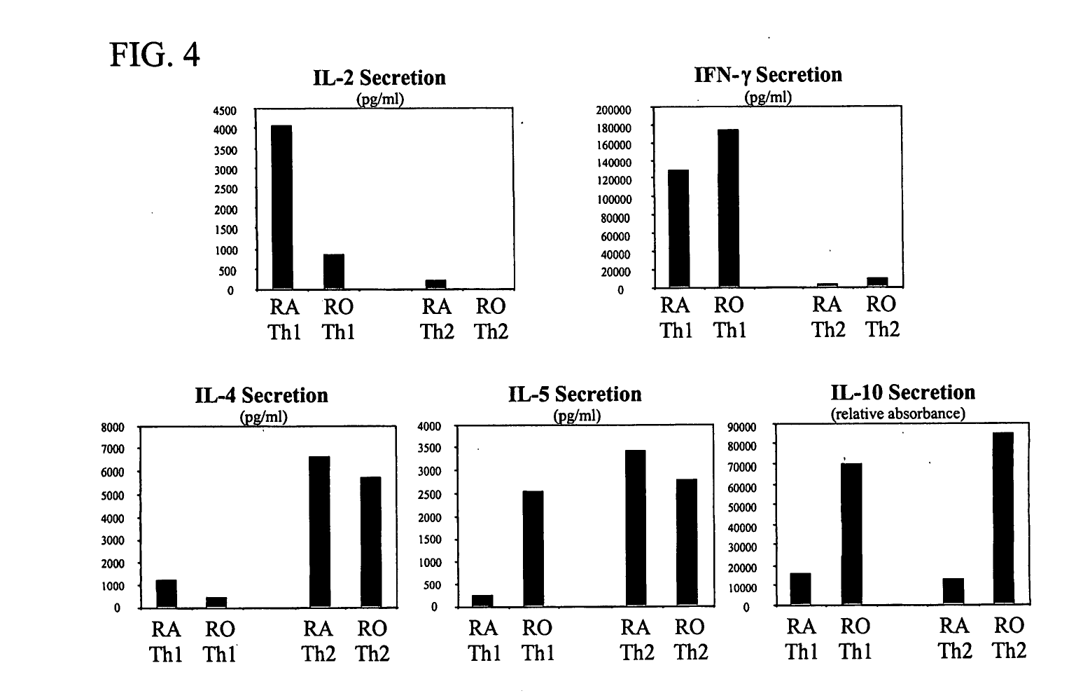

As shown in FIG. 4, the CD4⁺RA⁺ subset cultured in the Th1 supportive environment had higher Th1 purity relative to the CD4+ RO subset. That is, relative to the Th1 culture condition using CD4⁺RO⁺ cells, the CD4⁺RA⁺ Th1 culture increased secretion of the type I cytokine IL-2 and a comparable level of the type I cytokine IFN-γ. Furthermore, relative to the Th1 culture condition using CD4⁺RO⁺ cells, the CD4⁺RA⁺ Th1 culture demonstrates a reduced secretion of type II cytokines IL-5 and IL-10, and a comparable level secretion of the type II cytokine IL-4. Therefore, the RA subset generated a purer Th1 phenotype (increased Th1-type cytokine secretion and decreased Th2-type cytokine secretion). In addition, the Th1 cells generated from the CD4⁺RA⁺ starting cell population had a greatly enriched Th1 cytokine profile relative to the control Th2 cultures initiated from the RA⁺ or RO⁺ cell subsets.

These results demonstrate that generation of the Th1 subset can be enhanced by further purification of the CD4⁺RA⁺ subset of CD4 cells.

### EXAMPLE 11

### Pharmacokinetic and Immune Studies

The methods below describe how subjects can be monitored before, during, and after treatment.

Evaluation of Pre-transplant Induction Chemotherapy Cycles

Blood samples (10 cc in green-top heparinized tube) are drawn to evaluate the effects of immune depletion. This sample is drawn just prior to each cycle of induction chemotherapy (within six days of the next cycle). Experiments can include the use of flow cytometry to detect depletion of lymphoid versus myeloid subpopulations during induction chemotherapy.

Evaluation of Transplant Chemotherapy Preparative Regimen

Blood samples (10 cc in green-top heparinized tube) are drawn to evaluate the effects of the fludarabine and cyclophosphamide regimen on immune depletion in a subject. Timepoints for this aspect of the study are: 1) immediately prior to preparative regimen chemotherapy (day -6); and 2) just prior to the PBSCT (day 0). Experiments consist of flow cytometry to detect depletion of host lymphoid versus myeloid subpopulations in the peri-transplant period.

Evaluation of Type I Versus Type II Cytokine Effects Post-transplant

Blood samples (30 cc in green-top heparinized tubes, and 10 cc in serum collection tubes) are drawn once weekly at the following timepoints: prior to starting induction chemotherapy, prior to each induction chemotherapy cycle, and then each week after transplant administration for the first 100 days post-transplant. Samples are delivered to the lab to perform experiments to measure plasma levels, intracellular cytokine levels, and gene expression analysis of type I versus type II cytokines in the first 100 days post-transplant.

Evaluation of Immune Reconstitution Post-transplant

Blood (25 ml in heparinized tube) is evaluated for immune reconstitution post-transplant. Included is an evaluation of T cell receptor diversity post-transplant using a PCR-based assay. Samples are evaluated monthly for 3 months, and then every 3 months for the first two years post-transplant.

On Study Evaluation

Clinical blood tests (CBC with differential, electrolytes, liver and mineral panels): for induction chemotherapy period, day 1 and then twice per week; for inpatient period post-transplantation, daily; after discharge post-transplant, once per week Follow-up visits are at day 140, day 180, day 290, and day 365 post-transplant. Patients are followed every six months for one year, and then yearly until 5 years post-transplant.

Toxicity Criteria

The NCI Common Toxicity Criteria version 2.0 is used. This document can be found at the NIH website.

### EXAMPLE 12

### Pharmaceutical Compositions and Modes of Administration

Various delivery systems for administering the therapies disclosed herein are known, and include,but are not limited to, intradermal, intramuscular, intraperitoneal, intravenous, subcutaneous, intranasal, and oral routes. The compounds may be administered by any convenient route, for example by infusion or bolus injection, by absorption through epithelial or mucocutaneous linings (e.g., oral mucosa, rectal, vaginal and intestinal mucosa, etc.) and may be administered together with other biologically active agents. Administration can be systemic or local.

The present disclosure also provides pharmaceutical compositions which include a therapeutically effective amount of purified Th1 cells, alone or with a pharmaceutically acceptable carrier. Furthermore, the pharmaceutical compositions or methods of treatment can be administered in combination with other therapeutic treatments, such as chemotherapeutic agents and/or antimicrobial agents, or vaccines.

Delivery Systems

The pharmaceutically acceptable carriers useful herein are conventional. Remington's Pharmaceutical Sciences, by Martin, Mack Publishing Co., Easton, Pa., 15th Edition (1975), describes compositions and formulations suitable for pharmaceutical delivery of the purified Th1 cells herein disclosed. In general, the nature of the carrier will depend on the mode of administration being employed. For instance, parenteral formulations usually comprise injectable fluids that include pharmaceutically and physiologically acceptable fluids such as water, physiological saline, balanced salt solutions, aqueous dextrose, sesame oil, glycerol, ethanol, combinations thereof, or the like, as a vehicle. The carrier and composition can be sterile, and the formulation suits the mode of administration. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The amount of purified Th1 cells effective in the treatment of a particular disorder or condition will depend on the nature of the disorder or condition, and can be determined by standard clinical techniques. In addition, in vitro assays can be employed to identify optimal dosage ranges. The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems.

The disclosure also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

In view of the many possible embodiments to which the principles of our disclosure may be applied, it should be recognized that the illustrated embodiments are only particular examples of the disclosure and should not be taken as a limitation on the scope of the disclosure. Rather, the scope of the disclosure is in accord with the following claims. We therefore claim as our invention all that comes within the scope and spirit of these claims.

1. A method of producing a population of substantially purified CD4⁺ Th1 lymphocytes, comprising:
stimulating a population of substantially purified CD4⁺ T cells isolated from a subject by contacting the population with anti-CD3 monoclonal antibody and antibody that specifically binds to a T cell costimulatory molecule, in the presence of a Th1 supportive environment, thereby producing a population of substantially purified CD4⁺ Th1 lymphocytes which secrete a Th1 cytokine.

2. The method of claim 1, wherein the Th1 supportive environment comprises at least 20 IU/ml of IL-2 and a neutralizing amount of an IL-4 neutralizing agent.

3. The method of claim 2, wherein the Th1 supportive environment comprises at least 750 IU/ml of IL-2 and a neutralizing amount of an IL-4 neutralizing agent.

4. The method of claim 3, wherein the Th1 supportive environment comprises about 1000 IU/ml of IL-2 and a neutralizing amount of an IL-4 neutralizing agent.

5. The method of claim 2, wherein the Th1 supportive environment further comprises at least 1 ng/ml of IL-12.

6. The method of claim 5, wherein the Th1 supportive environment further comprises about 2.5 ng/ml of IL-12.

7. The method of claim 2, wherein the Th1 supportive environment further comprises a neutralizing amount of a IL-13 neutralizing agent.

8. The method of claim 2, wherein the Th1 supportive environment further comprises a neutralizing amount of a IL-4/IL-13 neutralizing agent.

9. The method of claim 5, wherein the Th1 supportive environment further comprises a neutralizing amount of a IL-13 neutralizing agent.

10. The method of claim 5, wherein the Th1 supportive environment further comprises a neutralizing amount of a IL-4/IL-13 neutralizing agent.

11. The method of claim 1, further comprising allowing the stimulated population of CD4⁺ T cells to proliferate in the Th1 supportive environment.

12. The method of claim 11, wherein the Th1 supportive environment comprises at least 20 IU/ml of IL-2 and a neutralizing amount of an IL-4/IL-13 neutralizing agent.

13. The method of claim 12, wherein the Th1 supportive environment comprises about 1000 IU/ml of IL-2 and a neutralizing amount of an IL-4/IL-13 neutralizing agent.

14. The method of claim 1, wherein the substantially purified CD4⁺ T cells are further purified into a CD4⁺RA⁺ T cell population.

15. The method of claim 1, wherein the Th1 cytokine is IL-2 or IFN-γ.

16. The method of claim 15, wherein the Th1 cytokine is IL-2.

17. The method of claim 1, wherein the population of substantially purified CD4⁺ Th1 lymphocytes comprises less than 5% Th2 lymphocytes.

18. The method of claim 17, wherein the population of substantially purified CD4⁺ Th1 lymphocytes comprises less than 1% Th2 lymphocytes.

19. The method of claim 1, wherein the population of substantially purified CD4⁺ Th1 lymphocytes produces less than 10 pg/ml of IL-4 per 1×10⁶ CD4⁺ Th1 lymphocytes.

20. The method of claim 1, wherein the population of substantially purified CD4⁺ Th1 lymphocytes produces at least 1000 pg/ml of IL-2 per 1×10⁶ CD4⁺ Th1 lymphocytes.

21. The method of claim 1, further comprising comparing the purity of the population of substantially purified CD4⁺ Th1 lymphocytes with a substantially purified population of purified CD4⁺ Th2 cells.

22. The method of claim 1, further comprising re-stimulating the substantially purified CD4⁺ Th1 lymphocytes with an immobilized anti-CD3 monoclonal antibody and an immobilized antibody that specifically binds to a T cell costimulatory molecule after allowing the cells to proliferate in the Th1 supportive environment.

23. The method of claim 2, wherein the IL-4 neutralizing agent is an anti-IL-4 antibody.

24. The method of claim 1, wherein the antibody that specifically binds to a T cell costimulatory receptor specifically binds CD28, inducible costimulatory molecule (ICOS), 4-1BB receptor (CDw137), lymphocyte function-associated antigen-1(LFA-1), CD30, or CD154.

25. The method of claim 24, wherein the antibody that specifically binds a T cell costimulatory molecule specifically binds CD28.

26. The method of claim 1, wherein the antibodies are immobilized.

27. The method of claim 26, wherein the immobilized anti-CD3 monoclonal antibody and the immobilized antibody that specifically binds a T cell costimulatory molecule are immobilized on a magnetic solid phase surface.

28. A CD4⁺ Th1 cell produced by the method of claim 1.

29. The method of claim 1, wherein the subject has at least one infectious disease.

30. The method of claim 1, wherein the subject has at least one tumor.

31. A method of producing a population of substantially purified CD4⁺ Th1 lymphocytes, comprising:
stimulating a population of substantially purified CD4⁺ T cells isolated from a subject by contacting the population with an immobilized anti-CD3 monoclonal antibody and an immobilized antibody that specifically binds to a T cell costimulatory molecule in the presence of a Th1 supportive environment, wherein the Th1 supportive environment comprises about 1000 IU/ml of IL-2, about 2.5 ng/ml IL-12, a neutralizing amount of an IL-4 neutralizing agent, and a neutralizing amount of an IL-13 neutralizing agent, thereby forming a stimulated population of T cells; and
allowing the stimulated population of CD4⁺ T cells to proliferate in a Th1 supportive environment comprising about 1000 IU/ml of IL-2, a neutralizing amount of an IL-4 neutralizing agent, and a neutralizing amount of an IL-13 neutralizing agent; thereby producing a population of substantially purified CD4⁺ Th1 lymphocytes, wherein the population of CD4⁺ Th1 lymphocytes secrete a Th1 cytokine.

32. A method of producing a population of substantially purified CD4⁺ Th1 lymphocytes, comprising:
obtaining a population of CD4⁺ T lymphocytes from a subject;
purifying a population of CD4⁺RA⁺ T cells from the CD4⁺ T lymphocytes;
initially stimulating the CD4⁺ T lymphocytes in a media comprising an anti-CD3 monoclonal antibody, an anti-CD28 monoclonal antibody, about 1000 IU/ml of IL-2, a neutralizing amount of an IL-4 neutralizing agent, and a neutralizing amount of an IL-13 neutralizing agent, wherein the anti-CD3 monoclonal antibody and the anti-CD28 monoclonal antibody are immobilized on a magnetized solid substrate; and
re-stimulating the T lymphocytes in the media, thereby producing a population of substantially purified CD4⁺ Th1 lymphocytes.

33. The method of claim 32, wherein the media further comprises about 2.5 ng/ml IL-12.

34. The method of claim 32, wherein the re-stimulation of the T-cells occurs within about eight to about twelve days of the initial stimulation of the T cells.

35. The method of claim 32, further comprising cryo-preserving the purified CD4⁺ Th1 lymphocytes.

36. A substantially purified population of CD4⁺ Th1 lymphocytes, wherein the population comprises less than 5% CD4⁺ Th2 lymphocytes.

37. The substantially purified population of CD4⁺ Th1 lymphocytes of claim 36, wherein the population comprises less than 1% CD4⁺ Th2 lymphocytes.

38. The substantially purified population of CD4⁺ Th1 lymphocytes of claim 36, wherein the population produces less than about 10 pg/ml of IL-4 per 1×10⁶ CD4^{+Th}1 lymphocytes.

39. The substantially purified population of CD4⁺ Th1 lymphocytes of claim 36, wherein the population produces at least 1000 pg/ml of IL-2 per 1×10⁶ CD4⁺ Th1 lymphocytes.

40. A method of enhancing an immune response, comprising:
administering to a subject a composition comprising a population of substantially purified CD4⁺ Th1 lymphocytes produced by the method of claim 1, wherein administration of the population of substantially purified CD4⁺ Th1 lymphocytes enhances the immune system of the subject.

41. The method of claim 40, wherein the population of substantially purified CD4⁺ Th1 lymphocytes are cryopreserved and thawed prior to administering the lymphocytes to the subject.

42. The method of claim 40, wherein the population of substantially purified CD4⁺ Th1 lymphocytes are administered at a dose of about 5×10⁶ to about 2×10⁸ substantially purified CD4⁺ Th1 lymphocytes per kilogram of subject.

43. The method of claim 40, wherein the composition is administered to treat an infectious disease.

44. The method of claim 43, wherein the infectious disease is a bacterial, viral, parasitic, or fungal infection.

45. The method of claim 40, wherein the composition further comprises a pharmaceutically acceptable carrier.

46. The method of claim 44, wherein the composition further comprises an anti-microbial agent.

47. The method of claim 40, wherein the composition further comprises non-cultured CD4⁺ and CD8⁺ T cells.

48. The method of claim 40, wherein the composition is administered to treat a tumor.

49. The method of claim 48, further comprising administering a cancer vaccine, chemotherapeutic agent, or a monoclonal antibody, to the subject.

50. A method of treating a subject having at least one tumor comprising:
producing a population of substantially purified CD4⁺ Th1 lymphocytes from the subject using the method of claim 1;
administering an immuno-depleting agent to the subject; and
administering the substantially purified CD4⁺ Th1 lymphocytes to the subject, wherein administration of the substantially purified CD4⁺ Th1 lymphocytes enhances the immune system of the subject.

51. The method of claim 50, wherein the immuno-depleting agent is a chemotherapeutic agent.

52. The method of claim 50, wherein the immuno-depleting agent is a monoclonal antibody.

53. A method of enhancing a vaccine response in a subject comprising:
administering a vaccine to the subject; and
administering to the subject a population of substantially purified CD4⁺ Th1 lymphocytes obtained using the method of claim 1, wherein administration of the substantially purified CD4⁺ Th1 lymphocytes enhances the vaccine response in the subject.

54. A method of transplanting immune cells to reconstitute immunity in a subject having a tumor, comprising:
immuno-depleting at least T cells in the subject;
administering to the subject a therapeutically effective amount of a population of autologous cells comprising CD4⁺ and CD8⁺ T cells; and
administering to the subject a therapeutically effective amount of a population of substantially purified CD4⁺ Th1 lymphocytes obtained using the method of claim 1, thereby transplanting autologous immune cells into the subject and reconstituting immunity in the subject.

55. The method of claim 54, wherein the population of autologous cells comprising CD4⁺ and CD8^{+T} cells are administered as a peripheral blood stem cell product.

56. The method of claim 54, wherein the therapeutically effective amount of a population of substantially purified CD4⁺ Th1 lymphocytes are obtained using the method of claim 11.

57. The method of claim 55, wherein the administration of autologous cells comprising CD4⁺ and CD8⁺ T cells, and the population of substantially purified CD4⁺ Th1 lymphocytes, is simultaneous.

58. The method of claim 54, wherein the population of substantially purified CD4⁺ Th1 lymphocytes are administered following the administration of the autologous cells comprising CD4⁺ and CD8⁺ T cells, within one day of the administration of the autologous cells comprising CD4⁺ and CD8⁺ T cells, and/or at a time remote from the administration of the autologous cells comprising CD4⁺ and CD8⁺ T cells.

59. The method of claim 54, wherein the population of substantially purified CD4⁺ Th1 lymphocytes are administered at a dose of about 5×10⁶ cells per kilogram to about 125×10⁶ cells per kilogram.

60. The method of claim 1, wherein the substantially purified CD4⁺ T cells are a CD4⁺RA⁺ T cell subset of CD4⁺ cells.
